(19) United States
(12) Patent Application Publication
(10) Pub. No.: US 2024/0398763 A1
(43) Pub. Date: Dec. 5, 2024

(54) ORAL DOSAGE FORM CONTAINING ONCOLOGICAL ACTIVE INGREDIENT

(71) Applicant: OWP Pharmaceuticals, Inc., Naperville, IL (US)

(72) Inventors: Paul Sudhakar, Shawnee, KS (US); Scott Boyer, West Chicago, IL (US)

(21) Appl. No.: 18/678,480

(22) Filed: May 30, 2024

Related U.S. Application Data
(60) Provisional application No. 63/512,005, filed on Jul. 5, 2023.
(60) Provisional application No. 63/504,812, filed on May 30, 2023.

(51) Int. Cl. A61K 31/4196; A61K 9/00; A61K 9/08; A61K 9/16; A61K 31/4535; A61K 31/7068; A61K 47/10; A61K 47/12; A61K 47/14; A61K 47/18; A61K 47/26

(52) U.S. Cl. CPC A61K 31/4196; A61K 9/0053; A61K 9/08; A61K 9/1617; A61K 9/1623; A61K 9/1652; A61K 31/4535; A61K 31/7068; A61K 47/10; A61K 47/12; A61K 47/14; A61K 47/183; A61K 47/26

(57) ABSTRACT

The present invention provides for a dosage form (e.g., liquid oral solution, liquid oral suspension, solid oral bead, granules, etc.) that contains one or more oncological active ingredients, methods of administering the same, and methods of manufacturing the same.

### CROSS-REFERENCE TO RELATED APPLICATIONS

This application claims the benefit of priority to U.S. Provisional Patent Application No. 63/504,812, filed May 30, 2023, which is hereby incorporated by reference herein.

### SUMMARY

The present invention provides for a dosage form (e.g., liquid oral solution, liquid oral suspension, solid oral bead, granules, etc.), capable of administration in divided doses. The dosage form contains one or more oncological active ingredients. With some patients (e.g., pediatric and/or geriatric), prescribing healthcare professionals may prefer to administer the oncological active ingredient (over a desired period of time) in an amount above or below the amount present in the solid dosage form. As such, the amount of oncological active ingredient can be titrated with the use of such dosage form. The present invention also provides for methods of administering the dosage form, and methods of manufacturing the same.

The present invention also provides for a solid oral bead containing capecitabine.

The present invention also provides for an oral liquid suspension containing raloxifene.

The present invention also provides for an oral liquid solution containing raloxifene.

The present invention also provides for an oral liquid suspension containing anastrozole.

The present invention also provides for an oral liquid solution containing anastrozole.

The present invention also provides for an oral liquid solution containing anastrozole.

The present invention also provides for an oral liquid suspension containing letrozole.

The present invention also provides for an oral liquid solution containing letrozole.

The present invention also provides for a solid oral bead containing (i) an active layer, (ii) an intermediate layer, (iii) enteric coating layer, and (iv) overcoating layer; wherein the active layer contains an oncological active ingredient as described herein.

The present invention also provides for an oral liquid solution containing (i) oncological active ingredient, (ii) preservative, (iii) buffer, pH control agent, (iv) sweetener-solvent, (v) solvent-vehicle, (vi) flavor, (vii) colorant, and (viii) chelating agent.

The present invention also provides for an oral liquid suspension containing (i) oncological active ingredient, (ii) preservative, (iii) buffer-pH control agent, (iv) sweetener-solvent, (v) solvent-vehicle, (vi) flavor, (vii) colorant, (viii) anticaking/suspending agent, (ix) viscosifying agent, and (x) taste masking agent.

The present invention also provides for an oral liquid solution containing: oncological active ingredient; buffer; sweetener; preservative; chelating agent; colorant; flavor; and solvent.

The present invention also provides for an oral liquid solution containing: oncological active ingredient (e.g., anastrozole); buffer (e.g., citric acid); sweetener (e.g., sorbitol; sodium saccharin); preservative (e.g., sodium benzoate; methylparaben); chelating agent (e.g., edetate disodium dihydrate); colorant (e.g., FD and C Red No. 40); flavor (e.g., cherry flavor, nat & art); and solvent (e.g., purified water).

The present invention also provides for an oral liquid solution containing: anastrozole; citric acid; sorbitol; sodium saccharin; sodium benzoate; methylparaben; edetate disodium dihydrate; FD and C Red No. 40; cherry flavor, nat & art; and purified water.

The present invention also provides for granules for an oral liquid suspension comprising: oncological active ingredient; buffer; sweetener; preservative; chelating agent; colorant; flavor (e.g., cherry flavor, nat & art); and solvent.

The present invention also provides for granules for an oral liquid suspension comprising: oncological active ingredient (e.g., capecitabine); buffer (e.g., citric acid); sweetener (e.g., sorbitol; sodium saccharin); preservative (e.g., sodium benzoate; methylparaben); chelating agent (e.g., edetate disodium dihydrate); colorant (e.g., FD and C Red No. 40); flavor (e.g., cherry flavor, nat & art); and solvent (e.g., purified water).

The present invention also provides for granules for an oral liquid suspension comprising: capecitabine; citric acid; sorbitol; sodium saccharin; sodium benzoate; methylparaben; edetate disodium dihydrate; FD and C Red No. 40; cherry flavor, nat & art; and purified water.

The present invention also provides for a liquid oral suspension comprising: oncological active ingredient; preservative; sweetener; buffer; cosolvent; solvent; anticaking; suspending agent; viscosifying agent; flavor; colorant; and taste masking agent.

The present invention also provides for a liquid oral suspension comprising: oncological active ingredient (e.g., letrozole); preservative (e.g., methylparaben; sodium benzoate); sweetener (e.g., saccharin sodium; sorbitol); buffer (e.g., sodium phosphate dibasic; sodium phosphate monobasic); cosolvent (e.g., propylene glycol); solvent (e.g., glycerin; purified water); anticaking (e.g., silicified microcrystalline cellulose); suspending agent (e.g., carboxymethylcellulose sodium); viscosifying agent (e.g., xanthan gum; polyethylene glycol 400); flavor (e.g., cherry flavor, nat & art); colorant (e.g., FD and C yellow No. 6); and taste masking agent (e.g., sucralose).

The present invention also provides for a liquid oral suspension comprising: letrozole; methylparaben; sodium benzoate; saccharin sodium; sorbitol; sodium phosphate dibasic; sodium phosphate monobasic; propylene glycol; glycerin; purified water; silicified microcrystalline cellulose; carboxymethylcellulose sodium; xanthan gum; polyethylene glycol 400; cherry flavor, nat & art; FD and C yellow No. 6; and sucralose.

The present invention also provides for a liquid oral suspension comprising: oncological active ingredient; preservative; sweetener; buffer; cosolvent; solvent; anticaking agent; suspending agent; viscosifying agent; flavor; colorant; and taste masking agent.

The present invention also provides for a liquid oral suspension comprising: oncological active ingredient (e.g., raloxifene hydrochloride); preservative (e.g., methylparaben; sodium benzoate); sweetener (e.g., saccharin sodium, dihydrate; sorbitol); buffer (e.g., sodium phosphate dibasic; sodium phosphate monobasic); cosolvent (e.g., propylene glycol); solvent (e.g., glycerin; purified water); anticaking agent (e.g., silicified microcrystalline cellulose); suspending agent (e.g., carboxymethylcellulose sodium, medium viscosity); viscosifying agent (e.g., xanthan gum; polyethylene glycol 400); flavor (e.g., cherry flavor, Nat & Art); colorant (e.g., FD and C Yellow No. 6); and taste masking agent (e.g., sucralose).

The present invention also provides for a liquid oral suspension comprising: raloxifene hydrochloride; methylparaben; sodium benzoate; saccharin sodium, dihydrate; sorbitol; sodium phosphate dibasic; sodium phosphate monobasic; propylene glycol; glycerin; purified water; silicified microcrystalline cellulose; carboxymethylcellulose sodium, medium viscosity; xanthan gum; polyethylene glycol 400; cherry flavor, Nat & Art; FD and C Yellow No. 6; and sucralose.

The present invention also provides for a liquid oral solution comprising: oncological active ingredient; buffer; sweetener; co-solvent; preservative; sweetener; colorant; flavor; and solvent.

The present invention also provides for a liquid oral solution comprising: oncological active ingredient (e.g., raloxifene hydrochloride); buffer (e.g., citric acid anhydrous; trisodium citrate dihydrate); sweetener (e.g., sorbitol); co-solvent (e.g., glycerin; propylene glycol); preservative (e.g., sodium benzoate); sweetener (e.g., sucralose); colorant (e.g., F&C Red No 40); flavor (e.g., cherry flavor); and solvent (e.g., purified water).

The present invention also provides for a liquid oral solution comprising: raloxifene hydrochloride; citric acid anhydrous; trisodium citrate dihydrate; sorbitol; glycerin; propylene glycol; sodium benzoate; sucralose; F&C Red No 40; cherry flavor; and purified water.

The present invention also provides for a liquid oral solution comprising: oncological active ingredient; buffer; sweetener; preservative; chelating agent; colorant; flavor; and solvent.

The present invention also provides for an liquid oral solution comprising: oncological active ingredient (e.g., anastrozole); buffer (e.g., citric acid anhydrous); sweetener (e.g., sorbitol; sodium saccharide); preservative (e.g., sodium benzoate; methylparaben); chelating agent (e.g., edetate disodium dihydrate); colorant (e.g., FD and C Red No. 40); flavor (e.g., cherry flavor); and solvent (e.g., purified water).

The present invention also provides for a liquid oral solution comprising: anastrozole; citric acid anhydrous; sorbitol; sodium saccharide; sodium benzoate; methylparaben; edetate disodium dihydrate; FD and C Red No. 40; cherry flavor; and purified water.

The present invention also provides for a liquid oral solution comprising: oncological active ingredient; buffer; sweetener; co-solvent; preservative; colorant; flavor; and solvent.

The present invention also provides for a liquid oral solution comprising: oncological active ingredient (e.g., letrozole); buffer (e.g., citric acid anhydrous; trisodium citrate dihydrate); sweetener (e.g., sorbitol; sucralose); co-solvent (e.g., glycerin; propylene glycol); preservative (e.g., sodium benzoate); colorant (e.g., F&C Red No 40); flavor (e.g., cherry flavor); and solvent (e.g., purified water).

The present invention also provides for a liquid oral solution comprising: letrozole; citric acid anhydrous; trisodium citrate dihydrate; sorbitol; sucralose; glycerin; propylene glycol; sodium benzoate; F&C Red No 40; cherry flavor; and purified water.

Suitable oncological active ingredients include, for example, anastrozole; capecitabine; letrozole; raloxifene hydrochloride; tretinoin; abemaciclib; abiraterone acetate; acalabrutinib; afatinib; avapritinib; everolimus; alectinib; melphalan; anastrozole; apalutamide; exemestane; axitinib; azacitidine; bexarotene; bicalutamide; bosutinib; brigatinib; busulfan; capecitabine; lomustine; cabozantinib; cabozantinib(S)-malate; vandetanib; etoposide; ceritinib; chlorambucil; lorlatinib; crizotinib; dabrafenib; trametinib; dasatinib; midostaurin; encorafenib; binimetinib; entrectinib; enzalutamide; erlotinib; letrozole; fludarabine phosphate; flutamide; tivozanib; gefitinib; imatinib mesylate; topotecan; hydroxyurca; ibandronate sodium; palbociclib; ibrutinib; ponatinib; ixazomib; lenalidomide; ribociclib; larotrectinib; lenvatinib; trifluridine; tipiracil hydrochloride; olaparib; mitotane; thalidomide; medroxyprogesterone acetate; methotrexate; neratinib; sorafenib tosylate; nilotinib; nintedanib; niraparib; osimertinib; panobinostat; pazopanib; pemigatinib; pomalidomide; temozolomide; idelalisib; raloxifene hydrochloride; regorafenib; recaparib; ruxolitinib phosphate; sunitinib malate; tamoxifen citrate; vemurafenib; venetoclax; erdafitinib; zanubrutinib; lomustine; duvelisib; cobimetinib fumarate; cyclophosphamide; glasdegib; estramustine phosphate; vismodegib; mobocertinib; flutamide; toremifene citrate; pralsetinib; enasidenib; decitabine; cedazuridine; fedratinib; selumetinib; adagrasib; sotorasib; futibatinib; mercaptopurine; nilutamide; darolutamide; sonidegib; relugolix; alpelisib; ripretinib; selpercatinib; olutasidenib; asciminib; thioguanine; capmatinib; talazoparib tosylate; tazemetostat; tepotinib; ivosidenib; infigratinib phosphate; tucatinib; pexidartinib; lapatinib; umbralisib tosylate; dacomitinib; pacritinib; belzutifan; gilteritinib; selinexor; abiraterone acetate; megestrol acetate; vorinostat; everolimus; conjugated estrogens (sodium estrone sulfate; sodium equilin sulfate); estradiol; levothyroxine sodium; ondansetron hydrochloride; and aprepitant.

### DETAILED DESCRIPTION

The present invention can be more readily understood by reading the following detailed description of the invention and study of the included examples.

As used herein, the following terms have the meanings ascribed to them unless specified otherwise.

The terms “comprise,” “comprising,” “include,” “including,” and “includes” when used in this specification and claims are intended to specify the presence of stated substances, features, integers, components, or steps, but they do not preclude the presence or addition of one or more other substances, features, integers, components, steps, or combinations thereof.

The term “about” modifies the subject values, such that they are within an acceptable error range, as determined by one of ordinary skill in the art, which will depend in part on the limitations of the measurement system.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

As used herein, “capecitabine” refers to the compound having the chemical name pentyl ester [1-(5-deoxy-β-d-ribofuranosyl)-5-fluoro-1,2-dihydro-2-oxo-4-pyrimidinyl]-carbamic acid; molecular formula C₁₅H₂₂FN₃O₆; and CAS Number 154361-50-9. Capecitabine is an antineoplastic agent, used to treat, e.g., breast cancer, gastric cancer, and colorectal cancer. The chemical structure is shown below.

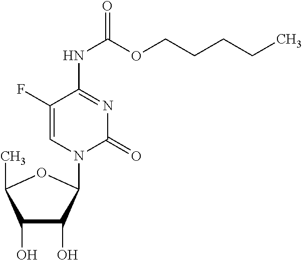

As used herein, “raloxifene” refers to the compound having the chemical name 6-hydroxy-2-(p-hydroxyphenyl)benzo[b]thien-3-yl-p-(2-piperidinoethoxy)phenyl ketone hydrochloride; molecular formula C₂₈H₂₇NO₄S•HCl; and CAS Number 82640 Apr. 8. Raloxifene is an estrogen agonist-antagonist (antiestrogen), used, e.g., in the treatment and prevention of osteoporosis in postmenopausal women. It is also used to reduce the risk of breast cancer in those at high risk. The chemical structure is shown below.

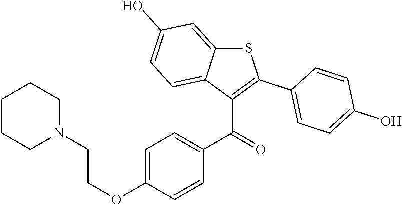 [SMILES: O=C(c1ccc(OCCN2CCCCC2)cc1)c1c(-c2ccc(O)cc2)sc2cc(O)ccc12]

As used herein, “anastrozole” refers to the compound with the chemical name α,α,α′,α′-tetramethyl-5-(1H-1,2,4-triazol-1-ylmethyl)-1,3-benzenediacetonitrile; molecular formula C₁₇H₁₉N₅; and CAS Number 120511-73-1. Anastrozole is an antiestrogen-aromatase inhibitor, used in addition to other treatments for breast cancer. The chemical structure is shown below.

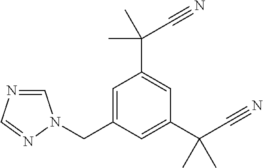 [SMILES: CC(C)(C#N)c1cc(Cn2cncn2)cc(C(C)(C)C#N)c1]

As used herein, “letrozole” refers to the compound with the chemical name 4,4′-(1H-1,2,4-triazol-1-ylmethylene)bis-benzonitrile; molecular formula C₁₇H₁₁N₅; and CAS Number 112809-51-5. Letrozole is an antineoplastic agent; selective aromatase inhibitor, used in the treatment of breast cancer. The chemical structure is shown below.

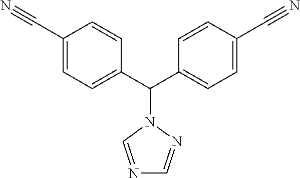 [SMILES: N#Cc1ccc(C(c2ccc(C#N)cc2)n2cncn2)cc1]

As used herein, “beads” refer to the solid oral dosage form of core-shell minispheres containing poorly water-soluble drugs or lipophilic drugs dissolved in the lipid core without the use of organic solvents and surfactants. Beads are typically prepared from cyclodextrins and oils. Beads with high drug loading and improved oral bioavailability have great potential for oral delivery of poorly water-soluble drugs and lipophilic drugs. Typically, beads are intended for enteral delivery by dissolving or suspending in a liquid beverage and orally administering, with or without food.

As used herein, “oral solution” refers to the oral liquid dosage form in which the active ingredient is mixed with a liquid, while being dissolved. The active ingredient particles are substantially dissolved in the liquid. Typically, oral solutions are intended for enteral delivery by orally administering, with or without food.

As used herein, “oral suspension” refers to the oral liquid dosage form in which the active ingredient is mixed with a liquid, but without being dissolved. The active ingredient particles are suspended in the liquid. The suspension should be shaken before use to diffuse the active ingredient particles uniformly in the liquid. Hence, optimum doses are essentially guaranteed. Typically, oral suspensions are intended for enteral delivery by orally administering, with or without food.

The dosage forms provided for herein may be prepared, e.g., by contacting the ingredient(s) with a solvent. In doing so, any one or more of the ingredients employed (active ingredient and/or excipients) can effectively be dissolved, suspended, or dispersed therein (e.g., in the solvent). This includes, e.g., salts, solvates, hydrates, dihydrates, monohydrates, powders, microcrystalline forms, crystalline forms, amorphous forms, specified particle size distribution (PSD), etc. of the ingredients. In doing so, the ingredient would therefore no longer necessarily retain that form. However, within the context of the present invention, it is appreciated that those of skill in the art understand and agree that reference to the resulting dosage form as containing ingredients in the indicated form is otherwise acceptable and appropriate.

Additionally, within the context of the present invention, it is appreciated that those of skill in the art understand and agree that reference to the dosage form as containing the ingredients having a specified state is acceptable and appropriate. This is so, even though those ingredients may no longer necessarily exist in the same state (as specifically indicated) as when introduced to the solvent. Likewise, within the context of the present invention, reference can also be made to the dosage form as being manufactured from (or as being formed from) the ingredients, as specifically indicated. It is appreciated that those of skill in the art understand and agree that each of the above characterizations of the dosage form are acceptable and appropriate.

For example, within the context of the dosage form, reference to “citric acid anhydrous” is also a reference to citric acid. Within the context of the dosage form, reference to “sorbitol solution, 70 percent” is also a reference to sorbitol. Within the context of the dosage form, reference to “sodium saccharin” or “saccharin sodium, dihydrate, powder” is also a reference to saccharin. Within the context of the dosage form, reference to “sodium benzoate, powder” is also a reference to benzoic acid and/or sodium benzoate. Within the context of the dosage form, reference to “methylparaben, powder” is also a reference to methylparaben. Within the context of the dosage form, reference to “edetate disodium dihydrate” is also a reference to edetate (EDTA). Within the context of the dosage form, reference to “carboxymethylcellulose sodium” is also a reference to carboxymethylcellulose sodium. Within the context of the dosage form, reference to “carboxymethylcellulose sodium, medium viscosity, viscosity of 2 percent aqueous solution @ 25 DEG C: 400-800 cP, USP” is also a reference to carboxymethylcellulose.

### Specific Embodiments

The specific embodiments provided below are for illustration purposes only, and do not otherwise limit the scope of the disclosed subject matter, as defined by the claims.

### Solid Oral Bead

In specific embodiments, the oral dosage form is a solid oral bead.

In specific embodiments, the oral dosage form is a solid oral bead containing capecitabine as an active ingredient.

In specific embodiments, the oral dosage form is a solid oral bead containing capecitabine as the sole active ingredient.

In specific embodiments, the oral dosage form is a solid oral bead having (i) an active layer, (ii) an intermediate layer, (iii) enteric coating layer, and (iv) overcoating layer.

In specific embodiments, the oral dosage form is a solid oral bead having (i) an active layer, (ii) an intermediate layer, (iii) enteric coating layer, and (iv) overcoating layer; wherein the active layer includes an active ingredient and at least one of sugar spheres, hypromellose 2910 5 cp, talc, and corn starch.

In specific embodiments, the oral dosage form is a solid oral bead having (i) an active layer, (ii) an intermediate layer, (iii) enteric coating layer, and (iv) overcoating layer; wherein the active layer includes capecitabine and at least one of sugar spheres, hypromellose 2910 5 cp, talc, and corn starch.

In specific embodiments, the oral dosage form is a solid oral bead having (i) an active layer, (ii) an intermediate layer, (iii) enteric coating layer, and (iv) overcoating layer; wherein the active layer includes (i) sugar spheres, (ii) capecitabine, (iii) hypromellose 2910 5 cp, (iv) talc, and (v) corn starch.

In specific embodiments, the oral dosage form is a solid oral bead having (i) an active layer, (ii) an intermediate layer, (iii) enteric coating layer, and (iv) overcoating layer; wherein the intermediate layer includes at least one of sucrose, talc, and hypromellose 2910 5 cp.

In specific embodiments, the oral dosage form is a solid oral bead having (i) an active layer, (ii) an intermediate layer, (iii) enteric coating layer, and (iv) overcoating layer; wherein the intermediate layer includes (i) sucrose, (ii) talc, and (iii) hypromellose 2910 5 cp.

In specific embodiments, the oral dosage form is a solid oral bead having (i) an active layer, (ii) an intermediate layer, (iii) enteric coating layer, and (iv) overcoating layer; wherein the enteric coating layer includes at least one of hypromellose phthalate, talc, and triethyl citrate.

In specific embodiments, the oral dosage form is a solid oral bead having (i) an active layer, (ii) an intermediate layer, (iii) enteric coating layer, and (iv) overcoating layer; wherein the enteric coating layer includes (i) hypromellose phthalate, (ii) talc, and (iii) triethyl citrate.

In specific embodiments, the oral dosage form is a solid oral bead having (i) an active layer, (ii) an intermediate layer, (iii) enteric coating layer, and (iv) overcoating layer; wherein the overcoating layer includes opadry AMB II high performance moisture barrier film coating 88A180040.

In specific embodiments, the solid oral bead containing capecitabine as an active ingredient, is dissolved or suspended in a liquid, and subsequently orally administered to a human subject.

In specific embodiments, the solid oral bead containing capecitabine as an active ingredient, is dissolved or suspended in a liquid, and subsequently orally administered to a human subject to treat breast cancer.

In specific embodiments, the solid oral bead containing capecitabine as an active ingredient, is dissolved or suspended in a liquid, and subsequently orally administered to a human subject to treat gastric cancer.

In specific embodiments, the solid oral bead containing capecitabine as an active ingredient, is dissolved or suspended in a liquid, and subsequently orally administered to a human subject to treat colorectal cancer.

### Oral Liquid Suspension

In specific embodiments, the oral dosage form is an oral liquid suspension.

In specific embodiments, the oral dosage form is an oral liquid suspension that includes raloxifene, anastrozole, or letrozole as an active ingredient.

In specific embodiments, the oral dosage form is an oral liquid suspension that includes raloxifene, anastrozole, or letrozole as the sole active ingredient.

In specific embodiments, the oral dosage form is an oral liquid suspension that includes raloxifene.

In specific embodiments, the oral dosage form is an oral liquid suspension that includes anastrozole.

In specific embodiments, the oral dosage form is an oral liquid suspension that includes letrozole.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the preservative includes at least one of betadex sulfobutyl ether sodium; carbon dioxide; betonite; chlorocresol; cresol; ethylparaben; imidurea; inactivation by magnesium trisilicate; lactic acid; parabens of varying alkyl chain lengths; phenol; phenylmercuric acetate; phenylmercuric borate; phenylmercuric nitrate; potassium sorbate; propionic acid; propylparaben; sodium borate; sodium lactate; sodium propionate; thimerosal; and xylitol.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the preservative is an antibacterial agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the preservative is an antibacterial agent that is at least one of acetic acid, glacial; diazolidinyl urea; dimethyl sulfoxide; iodine/edetic acid; phenylmercuric hydroxide; potassium sorbate; sodium hydroxide; and sorbic acid.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the preservative is an antimicrobial preservative.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the preservative is an antimicrobial preservative that is at least one of acetone sodium bisulfite; alcohol; benzalkonium chloride; benzethonium chloride; benzoic acid; benzyl alcohol; benzyl benzoate; betadex sulfobutyl ether sodium; boric acid; bronopol; butylated hydroxyanisole; butylene glycol; butylparaben; calcium acetate; calcium chloride; calcium lactate; cetrimide; cetromonium bromide; cetylpyridinium chloride; chlorhexidine; chlorobutanol; chloroxylenol; cresol; ethylparaben; glycerin; hexetidine; hydrogen peroxide; isopropyl alcohol; monothioglycerol; pentetic acid; phenol; phenoxyethanol; phosphoric acid; potassium benzoate; potassium metabisulfite; potassium nitrate; potassium sorbate; providone-iodine; propyl gallate; propylene glycol; propylparaben sodium; sodium acetate; sodium benzoate; sodium bisulfite; sodium borate; sodium lactate; sodium metabisulfite; sodium sulfite; sorbic acid; tetrasodium edtate; thimerasol; and zinc oxide.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the preservative is methylparaben and sodium benzoate.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the buffer (pH control agent) is at least one of ammonium phosphate; ammonium sulfate; arginine; calcium carbonate; calcium lactate; calcium phosphate, tribasic; diethanolamine; glycine; glycine hydrochloride; histidine; lysine acetate; lysine hydrochloride; meglumine; monoethanolamine; potassium citrate; potassium metaphosphate; potassium phosphate, dibasic; sodium acetate; sodium borate; sodium carbonate; sodium citrate; sodium lactate; sodium phosphate, dibasic; sodium phosphate, monobasic; sodium phosphate, tribasic; and tromethamine.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the buffer (pH control agent) includes sodium phosphate dibasic and sodium phosphate monobasic.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the solvent-sweetener is a sweetening agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the solvent-sweetener is a sweetening agent that is at least one of acesulfame potassium; alitame; ammonium glycyrrhizate; aspartame; compressible sugar; confectioner's sugar; corn syrup solids; dextrose; dextrose anhydrous; erythritol; fructose; galactose; glycerin; glycine; glycyrrhizin; inulin; invert sugar; isomalt; lactitol; liquid glucose; maltitol; maltitol solution; maltose; mannitol; D-mannose; neohesperidin dihydrochalcone; neotame; saccharin; saccharin calcium; saccharin sodium; sodium cyclamate; sorbitol; sucralose; sucrose; tagatose; thaumatin; trehalose; and xylitol.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the solvent-vehicle is a solvent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the solvent-vehicle is a solvent that is at least one of acetone; albumin; alcohol; almond oil; benzyl alcohol; benzyl benzoate; butylene glycol; carbon dioxide; castor oil; corn oil (maize); cottonseed oil; dibutyl phthalate; diethyl phthalate; diethylene glycol; dimethyl ether; dimethyl phthalate; dimethyl sulfoxide; dimethylacetamide; dipropyleneglycol; ethyl acetate; ethyl lactate; ethyl oleate; glycerin; glyceryl monostearate; glycofurol; n-hexane; isopropyl alcohol; isopropyl myristate; isopropyl palmitate; light mineral oil; medium-chain triglycerides; methyl lactate; methylpyrrolidone; mineral oil; monothanolamine; octyldodecanol; olive oil; peanut oil; polyethylene glycol; polyoxyl 35 castor oil; propylene carbonate; propylene glycol; propylene glycol dicarpylate/dicaprate; pyrrolidone; safflower oil; sesame oil; soybean oil; sunflower oil; triacetin; tricaprylin; tricthanolamine; tricthyl citrate; triolein; and water.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the solvent-vehicle is a solubilizing agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the solvent-vehicle is a solubilizing agent that is at least one of anionic emulsifying wax, arginine hydrochloride; benzalkonium chloride; benzethonium chloride; betadex sulfobutyl ether sodium; cetylpyridinium chloride; cholestyramine resin; cyclodextrins; diethylene glycol; dimethyl-β-cyclodextrin; fumaric acid; glycerin; glyceryl laurate; glyceryl monolinoleate; glycerol monostearate; glyceryl tricaprylate; hydroxpropyl betadex; hydroxyethyl-β-cyclodextrin; hypromellose; hypromellose acetate succinate; hypromellose phthalate; lanolin alcohols; lecithin; linoleic acid; meglumine; methylpyrrolidone; niacinamide; nonionic emulsifying wax; oleic acid; oleyl alcohol; phospholipids; polacrilex resin; polacrillin resin; poloxamer; polymethcrylates; polyoxyethylene alkyl ethers; polyoxyelthylene castor oil derivatives; polyoxyethylene sobitan fatty acid esters; polyoxyl 15 hydroxystearate; polyoxylglycerides; polyvinyl acetate phthalate; povidone; propylene glycol dicaprylate/dicaprate; propylene glycol dilaurate; propylene glycol monocaprylate; propylene glycol monolaurate; pyrrolidone; silicon dioxide, hydrated; sodium bicarbonate; sodium lauryl sulfate; sodium polystyrene sulfonate; sorbitan esters; stearic acid; sucrose palmitate; sucrose stearate; tricaprylin; trimethyl-β-cyclodextrin; triolein; vitamin E polyethylene glycol succinate; wax, anionic, emulsifying; and wax, nonanionic, emulsifying.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the solvent-vehicle includes at least one of propylene glycol, glycerin, and water.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the flavor is a flavor enhancer.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the flavor is a flavor enhancer that includes at least one of acesulfame potassium; aspartame; corn syrup, solids; ethyl maltol; ethylcellulose; fructose; matol; monosodium glutamate; neohesperidin dihydrochalcone; neotame; polydextrose; saccharin; saccharin sodium; silicon dioxide, hydrated; sodium cyclamate; thaumatin; trehalose; and xylitol.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the flavor is a flavoring agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the flavor is a flavoring agent that is at least one of adipic acid; ammonium glycyrrhizate; benzyl benzoate; n-butyl lactate; capric acid; confectioner's sugar; denatonium benzoate; diethyl sebacate; ethyl acetate; ethyl lactate; ethyl maltol; ethyl vanillin; fructose; fumaric acid; glycyrrhizin; leucine; malic acid; maltol; menthol; methionine; phenylethyl alcohol; phosphoric acid; saccharin calcium; saccharin sodium; sodium acetate; sodium lactate; sodium propionate; sucrose octaacetate; tartaric acid; thymol; and vanillin.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the flavor includes cherry flavor.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the colorant is a coloring agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the colorant is a coloring agent that is at least one of brilliant blue FCF; indigotine; alphazurine FG; indanthrene blue; fast green FCF; alizarin cyanine green F; quinizarin green SS; pyranine concentrated; Orange II; dibromofluorescein; diiodofluorescein; erythrosine yellowish Na; erythrosine; Ponceau SX; lithol rubin B; lithol rubin B Ca; toney red; tetrabromofluroescein; cosine; tetrachlorotetrabromofluroescein; phloxine B; helindone pink CN; brilliant lake red R; acid fuchsine; lake bordeaux B; flaming red; alba red; allura red AC; Allura Red AC; alizurol purple SS; tartrazine; sunset yellow FCF; fluorescein; naphthol yellow S; uranine; quinoline yellow WS; quinoline yellow SS; FD&C lakes; D&C lakes; Ext. D&C lakes; alumina; aluminum powder; annatto extract; beta-carotene; bismuth oxychloride; bronze powder; calcium carbonate; canthaxanthin; caramel; chromium-colbalt-aluminum oxide; chromium hydroxide green; chromium oxide green; cochincal extract, carmine; copper powder; dihydroxyaceteone; ferric ammonium citrate; ferric ammonium ferrocyanide; ferric ferrocynaide; guanine; iron oxides synthetic; logwood extract; mica; mica-based pearlescent pigments; potassium sodium copper chlorophyllin; pryogallol; pyrophyllite; talc; titanium dioxide; and zinc oxide.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the colorant is a coloring agent that includes FD&C Red No. 40; allura red AC; and FD&C Yellow No. 6.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the anticaking/suspending agent includes an anticaking agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the anticaking/suspending agent includes an anticaking agent that is at least one of calcium phosphate, tribasic; calcium silicate; cellulose, powdered; magnesium oxide; magnesium silicate; magnesium trisilicate; maltodextrin; silicon dioxide; silicon dioxide anhydrous; silicon dioxide, hydrated; silicon dioxide, hydrophobic; and talc.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the anticaking/suspending agent includes a suspending agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the anticaking/suspending agent includes a suspending agent that is at least one of acacia; agar; alginic acid; attapulgite; bentonite; calcium stearate; carbomers; carboxymethylcellulose calcium; caboxymethylcellulose sodium; microcrystalline cellulose; carrageenan; cellulose microcrystalline, and carboxymethylcellulose sodium, powdered; ceratonia; corn syrup solids; dextrin; gelatin; guar gum; hectorite; hydroxyethyl cellulose; hydrxyethylmethyl cellulose; hydroxypropyl cellulose; hypromellose; isomalt; kaolin; magnesium aluminum silicate; maltitol solution; medium-chain triglycerides; methylcellulose; phospholipids; poloxamer; polycarbophil; polyethylene glycol; polyoxyethylene sorbitan fatty acid esters; polyvinyl acetate phthalate; potassium alginate; povidone; propylene glycol alginate; saponite; sesame oil; silicon dioxide, anhydrous; silicon dixoide, hydrated; silicon dioxide, hydrophobic; sodium alginate; sorbitan esters; sucrose; tragacanath; vitamin E polyethylene glycol succinate; and xanthan gum.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the anticaking/suspending agent includes Vivapur MCG 591P.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the viscosifying agent includes a viscosity-enhancing agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the viscosifying agent includes a viscosity-enhancing agent that is at least one of acacia; agar; alginic acid; aluminum monostearate; ammonium sulfate; attapulgite; bentonie; betadex sulfobutyl ether sodium; calcium alginate; calcium lactate; carbomers; carobyxmethylcellulose calcium; carobyxmethylcellulose sodium and microcrystalline cellulose; carrageenan; cellulose; ceratonia; ceresin; cetostearyl alcohol; cetyl palmitate; chitosan; corn syrup solids; cyclomethicone; dextrin; ethylcellulose; gelatin; gellan gum; glycerin; glyceryl bchenate; glyceryl laurate; guar gum; hectorite; hydrogenated palm oil; hydrogenated vegetable oil type I, hydroxyethyl cellulose; hydroxypropyl cellulose; hypromellose; invert sugar; magnesium aluminum silicate; maltodesxtrin; methylcellulose; modified starch; myristyl alcohol; octyldodecanol; pectin; polycarbophil; polydextrose; polyethylene glycol; polyethylene oxide; poly(methylvinyl ether/maleic anhydride); polyoxyethylene alkyl ethers; polyvinyl alcohol; potassium alginate; povidone; propylene glycol alginate; propylene glycol dilaurate; pullulan; saponite; silicon dioxide anhydrous, silicon dioxide hydrophobic; sodium alginate; sodium chloride; starch; stearic acid; stearyl alcohol; sucrose; tragacanth; trehalose; and xanthan gum.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the viscosifying agent includes a thickening agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the viscosifying agent includes a thickening agent that is at least one of alginic acid; ammonium alginate; ceresin; dextrin; ethylcellulose; ethylene glycol palmitostearate; hectorite; methylcellulose; modified starch; polyethylene glycol; potassium chloride; silicon dioxide, anhydrous; xanthan gum; and zinc stearate.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the viscosifying agent includes a stiffening agent.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the viscosifying agent includes a stiffening agent that is at least one of anionic emulsifying wax; carnauba wax; ceresin; cetyl alcohol; cetyl esters wax; cetyl palmitate; dextrin; hydrogenated castor oil; microcrystalline wax; nonionic emulsifying wax; paraffin; sodium stearate; stearyl alcohol; white wax; and yellow wax.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the viscosifying agent includes xanthan gum and polyethylene glycol 400.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the taste masking agent includes at least one of alginic acid; ammonium glycyrrhizate; cellulose acetate; cholestyramine resin; erythritol; ethylcellulose; fructose; fructose and pregelatinized starch, coprocessed; glyceryl behenate; glyceryl palmitostearate; glycyrrhizin; hypromellose phthalate; invert sugar; isomalt; polacrilin potassium; polacrilin resin; polydextrose; saccharin calcium; shellac; silicon dioxide, hydrated; sodium polystyrene sulfonate; and xylitol.

In specific embodiments, the oral dosage form is an oral liquid suspension that includes an active ingredient (e.g., raloxifene, anastrozole, or letrozole), a preservative, buffer (pH control agent), sweetener-solvent, solvent-vehicle, flavor, colorant, anticaking/suspending agent, viscosifying agent, and taste masking agent; wherein the taste masking agent includes sucralose.

In specific embodiments, the oral liquid suspension that includes raloxifene as an active ingredient is orally administered to a human subject.

In specific embodiments, the oral liquid suspension that includes raloxifene as an active ingredient is orally administered to a human subject for the treatment and prevention of osteoporosis in postmenopausal women.

In specific embodiments, the oral liquid suspension that includes raloxifene as an active ingredient is orally administered to a human subject to reduce the risk of breast cancer in those at high risk.

In specific embodiments, the oral liquid suspension that includes anastrozole as an active ingredient is orally administered to a human subject in addition to other treatments for the treatment of breast cancer.

In specific embodiments, the oral liquid suspension that includes letrozole as an active ingredient is orally administered to a human subject for the treatment of breast cancer.

### Oral Liquid Solution

In specific embodiments, the oral dosage form is an oral liquid solution.

In specific embodiments, the oral dosage form is an oral liquid solution that includes raloxifene, anastrozole, or letrozole as an active ingredient.

In specific embodiments, the oral dosage form is an oral liquid solution that includes raloxifene, anastrozole, or letrozole as the sole active ingredient.

In specific embodiments, the oral dosage form is an oral liquid solution that includes raloxifene.

In specific embodiments, the oral dosage form is an oral liquid solution that includes anastrozole.

In specific embodiments, the oral dosage form is an oral liquid solution that includes letrozole.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the preservative includes at least one of betadex sulfobutyl ether sodium; carbon dioxide; betonite; chlorocresol; cresol; ethylparaben; imidurea; inactivation by magnesium trisilicate; lactic acid; parabens of varying alkyl chain lengths; phenol; phenylmercuric acetate; phenylmercuric borate; phenylmercuric nitrate; potassium sorbate; propionic acid; propylparaben; sodium borate; sodium lactate; sodium propionate; thimerosal; and xylitol.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the preservative is an antibacterial agent.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the preservative is an antibacterial agent that is at least one of acetic acid, glacial; diazolidinyl urea; dimethyl sulfoxide; iodine/edetic acid; phenylmercuric hydroxide; potassium sorbate; sodium hydroxide; and sorbic acid.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the preservative is an antimicrobial preservative.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the preservative is an antimicrobial preservative that is at least one of acetone sodium bisulfite; alcohol; benzalkonium chloride; benzethonium chloride; benzoic acid; benzyl alcohol; benzyl benzoate; betadex sulfobutyl ether sodium; boric acid; bronopol; butylated hydroxyanisole; butylene glycol; butylparaben; calcium acetate; calcium chloride; calcium lactate; cetrimide; cetromonium bromide; cetylpyridinium chloride; chlorhexidine; chlorobutanol; chloroxylenol; cresol; ethylparaben; glycerin; hexetidine; hydrogen peroxide; isopropyl alcohol; monothioglycerol; pentetic acid; phenol; phenoxyethanol; phosphoric acid; potassium benzoate; potassium metabisulfite; potassium nitrate; potassium sorbate; providone-iodine; propyl gallate; propylene glycol; propylparaben sodium; sodium acetate; sodium benzoate; sodium bisulfite; sodium borate; sodium lactate; sodium metabisulfite; sodium sulfite; sorbic acid; tetrasodium cdtate; thimerasol; and zinc oxide.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the preservative is methylparaben and sodium benzoate.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the buffer (pH control agent) is at least one of ammonium phosphate; ammonium sulfate; arginine; calcium carbonate; calcium lactate; calcium phosphate, tribasic; diethanolamine; glycine; glycine hydrochloride; histidine; lysine acetate; lysine hydrochloride; meglumine; monoethanolamine; potassium citrate; potassium metaphosphate; potassium phosphate, dibasic; sodium acetate; sodium borate; sodium carbonate; sodium citrate; sodium lactate; sodium phosphate, dibasic; sodium phosphate, monobasic; sodium phosphate, tribasic; and tromethamine.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the buffer (pH control agent) includes sodium phosphate dibasic and sodium phosphate monobasic.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the solvent-sweetener is a sweetening agent.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the solvent-sweetener is a sweetening agent that is at least one of acesulfame potassium; alitame; ammonium glycyrrhizate; aspartame; compressible sugar; confectioner's sugar; corn syrup solids; dextrose; dextrose anhydrous; erythritol; fructose; galactose; glycerin; glycine; glycyrrhizin; inulin; invert sugar; isomalt; lactitol; liquid glucose; maltitol; maltitol solution; maltose; mannitol; D-mannose; neohesperidin dihydrochalcone; neotame; saccharin; saccharin calcium; saccharin sodium; sodium cyclamate; sorbitol; sucralose; sucrose; tagatose; thaumatin; trehalose; and xylitol.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the solvent-vehicle is a solvent.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the solvent-vehicle is a solvent that is at least one of acetone; albumin; alcohol; almond oil; benzyl alcohol; benzyl benzoate; butylene glycol; carbon dioxide; castor oil; corn oil (maize); cottonseed oil; dibutyl phthalate; diethyl phthalate; diethylene glycol; dimethyl ether; dimethyl phthalate; dimethyl sulfoxide; dimethylacetamide; dipropyleneglycol; ethyl acetate; ethyl lactate; ethyl oleate; glycerin; glyceryl monostearate; glycofurol; n-hexane; isopropyl alcohol; isopropyl myristate; isopropyl palmitate; light mineral oil; medium-chain triglycerides; methyl lactate; methylpyrrolidone; mineral oil; monothanolamine; octyldodecanol; olive oil; peanut oil; polyethylene glycol; polyoxyl 35 castor oil; propylene carbonate; propylene glycol; propylene glycol dicarpylate/dicaprate; pyrrolidone; safflower oil; sesame oil; soybean oil; sunflower oil; triacetin; tricaprylin; triethanolamine; triethyl citrate; triolein; and water.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the solvent-vehicle is a solubilizing agent.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the solvent-vehicle is a solubilizing agent that is at least one of anionic emulsifying wax, arginine hydrochloride; benzalkonium chloride; benzethonium chloride; betadex sulfobutyl ether sodium; cetylpyridinium chloride; cholestyramine resin; cyclodextrins; diethylene glycol; dimethyl-β-cyclodextrin; fumaric acid; glycerin; glyceryl laurate; glyceryl monolinoleate; glycerol monostearate; glyceryl tricaprylate; hydroxpropyl betadex; hydroxyethyl-β-cyclodextrin; hypromellose; hypromellose acetate succinate; hypromellose phthalate; lanolin alcohols; lecithin; linoleic acid; meglumine; methylpyrrolidone; niacinamide; nonionic emulsifying wax; oleic acid; olcyl alcohol; phospholipids; polacrilex resin; polacrillin resin; poloxamer; polymethcrylates; polyoxyethylene alkyl ethers; polyoxyelthylene castor oil derivatives; polyoxyethylene sobitan fatty acid esters; polyoxyl 15 hydroxystearate; polyoxylglycerides; polyvinyl acetate phthalate; povidone; propylene glycol dicaprylate/dicaprate; propylene glycol dilaurate; propylene glycol monocaprylate; propylene glycol monolaurate; pyrrolidone; silicon dioxide, hydrated; sodium bicarbonate; sodium lauryl sulfate; sodium polystyrene sulfonate; sorbitan esters; stearic acid; sucrose palmitate; sucrose stearate; tricaprylin; trimethyl-β-cyclodextrin; triolein; vitamin E polyethylene glycol succinate; wax, anionic, emulsifying; and wax, nonanionic, emulsifying.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the solvent-vehicle includes at least one of propylene glycol, glycerin, and water.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the flavor is a flavor enhancer.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the flavor is a flavor enhancer that includes at least one of acesulfame potassium; aspartame; corn syrup, solids; ethyl maltol; ethylcellulose; fructose; matol; monosodium glutamate; neohesperidin dihydrochalcone; neotame; polydextrose; saccharin; saccharin sodium; silicon dioxide, hydrated; sodium cyclamate; thaumatin; trehalose; and xylitol.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the flavor is a flavoring agent.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the flavor is a flavoring agent that is at least one of adipic acid; ammonium glycyrrhizate; benzyl benzoate; n-butyl lactate; capric acid; confectioner's sugar; denatonium benzoate; diethyl sebacate; ethyl acetate; ethyl lactate; ethyl maltol; ethyl vanillin; fructose; fumaric acid; glycyrrhizin; leucine; malic acid; maltol; menthol; methionine; phenylethyl alcohol; phosphoric acid; saccharin calcium; saccharin sodium; sodium acetate; sodium lactate; sodium propionate; sucrose octaacetate; tartaric acid; thymol; and vanillin.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the flavor includes cherry flavor.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the colorant is a coloring agent.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the colorant is a coloring agent that is at least one of brilliant blue FCF; indigotine; alphazurine FG; indanthrene blue; fast green FCF; alizarin cyanine green F; quinizarin green SS; pyranine concentrated; Orange II; dibromofluorescein; diiodofluorescein; erythrosine yellowish Na; erythrosine; Ponceau SX; lithol rubin B; lithol rubin B Ca; toney red; tetrabromofluroescein; cosine; tetrachlorotetrabromofluroescein; phloxine B; helindone pink CN; brilliant lake red R; acid fuchsine; lake bordeaux B; flaming red; alba red; allura red AC; Allura Red AC; alizurol purple SS; tartrazine; sunset yellow FCF; fluorescein; naphthol yellow S; uranine; quinoline yellow WS; quinoline yellow SS; FD&C lakes; D&C lakes; Ext. D&C lakes; alumina; aluminum powder; annatto extract; beta-carotene; bismuth oxychloride; bronze powder; calcium carbonate; canthaxanthin; caramel; chromium-colbalt-aluminum oxide; chromium hydroxide green; chromium oxide green; cochincal extract, carmine; copper powder; dihydroxyacetcone; ferric ammonium citrate; ferric ammonium ferrocyanide; ferric ferrocynaide; guanine; iron oxides synthetic; logwood extract; mica; mica-based pearlescent pigments; potassium sodium copper chlorophyllin; pryogallol; pyrophyllite; talc; titanium dioxide; and zinc oxide.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the colorant is a coloring agent that includes FD&C Red No. 40; allura red AC; and FD&C Yellow No. 6.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the chelating agent includes a complexing agent.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the chelating agent includes a complexing agent that is at least one of antioxidants; betadex sulfobutyl ether sodium; calcium acetate; cholestyramine resin; citric acid monohydrate; cyclodextrins; dibasic sodium phosphate; dimethyl-β-cyclodextrin; edetate calcium disodium; edetate disodium; edetic acid; fumaric acid; galactose; gelatin; gluconolactone; hydroxpropyl betadex; hydroxyethyl-β-cyclodextrin; maltol; monobasic sodium phosphate; phosphoric acid; polacrilin potassium; polacrilin resin; poly(methylvinyl ether/maleic anhydride); potassium citrate; sodium citrate dihydrate; sodium polystyrene sulfonate; tartaric acid; tetrasodium edetate; trehalose; trimethyl-β-cyclodextrin; trisodium edetate; and zinc oxide.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the chelating agent includes an antioxidant.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the chelating agent includes an antioxidant that is at least one of acetone sodium bisulfite; alpha tocopherol; ascorbic acid; ascorbyl palmitate; butylated hydroxyanisole; butylated hydroxytoluene; carbon dioxide; chelating agents; citric acid monohydrate; cysteine; cysteine hydrochoride; dodecyl gallate; erythorbic acid; ethyl oleate; fumaric acid; histidine; malic acid; D-mannose; monothioglycerol; niacinamide; octyl gallate; phosphoric acid; potassium metabisulfite; propionic acid; propyl gallate; sodium ascorbate; sodium bisulfite; sodium formaldehyde sulfoxylate; sodium metabisulfite; sodium sulfite; sodiumthiosulfate; sulfur dioxide; tartaric acid; thymol; vitamin E polyethylene glycol succinate; and tocopherol.

In specific embodiments, the oral dosage form is an oral liquid solution that includes an active ingredient (e.g., raloxifene, anastrozole, or letrozole), a preservative, buffer (pH control agent), sweetener-solvent, solvent-vehicle, flavor, colorant, and chelating agent; wherein the chelating agent includes edetate disodium dihydrate.

In specific embodiments, the oral liquid solution that includes raloxifene as an active ingredient is orally administered to a human subject.

In specific embodiments, the oral liquid solution that includes raloxifene as an active ingredient is orally administered to a human subject for the treatment and prevention of osteoporosis in postmenopausal women.

In specific embodiments, the oral liquid solution that includes raloxifene as an active ingredient is orally administered to a human subject to reduce the risk of breast cancer in those at high risk.

In specific embodiments, the oral liquid solution that includes anastrozole as an active ingredient is orally administered to a human subject in addition to other treatments for the treatment of breast cancer.

In specific embodiments, the oral liquid solution that includes letrozole as an active ingredient is orally administered to a human subject for the treatment of breast cancer.

In specific embodiments, the oral dosage form is an oral liquid solution that contains one or more oncological active ingredients provided in Table 1 (Example 8).

In specific embodiments, the oral dosage form is an oral liquid solution that contains one or more oncological active ingredient, in the specified strength(s), as provided in Table 1 (Example 8).

In specific embodiments, the oral dosage form is an oral liquid solution that contains one or more oncological active ingredient, for the indication(s), as provided in Table 1 (Example 8).

In specific embodiments, the oral dosage form is an oral liquid solution that contains one or more oncological active ingredient, in the specified strength(s) and for the indication(s), as provided in Table 1 (Example 8).

In specific embodiments, the oral dosage form is granules that contain one or more oncological active ingredient provided in Table 1 (Example 8).

In specific embodiments, the oral dosage form is granules that contain one or more oncological active ingredient, in the specified strength(s), as provided in Table 1 (Example 8).

In specific embodiments, the oral dosage form is granules that contain one or more oncological active ingredient, for the indication(s), as provided in Table 1 (Example 8).

In specific embodiments, the oral dosage form is granules that contain one or more oncological active ingredient, in the specified strength(s) and for the indication(s), as provided in Table 1 (Example 8).

In specific embodiments, the oral dosage form is an oral liquid suspension that contains one or more oncological active ingredient provided in Table 1 (Example 8).

In specific embodiments, the oral dosage form is an oral liquid suspension that contains one or more oncological active ingredient, in the specified strength(s), as provided in Table 1 (Example 8).

In specific embodiments, the oral dosage form is an oral liquid suspension that contains one or more oncological active ingredient, for the indication(s), as provided in Table 1 (Example 8).

In specific embodiments, the oral dosage form is an oral liquid suspension that contains one or more oncological active ingredient, in the specified strength(s) and for the indication(s), as provided in Table 1 (Example 8).

### Enumerated Embodiments

Specific enumerated embodiments <1> to <34> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

### Embodiment 1

A solid oral bead containing capecitabine.

### Embodiment 2

An oral liquid suspension containing raloxifene.

### Embodiment 3

An oral liquid solution containing raloxifene.

### Embodiment 4

An oral liquid suspension containing anastrozole.

### Embodiment 5

An oral liquid solution containing anastrozole.

### Embodiment 6

An oral liquid suspension containing letrozole.

### Embodiment 7

An oral liquid solution containing letrozole.

### Embodiment 8

The solid oral bead of Embodiment <1>, containing (i) an active layer, (ii) an intermediate layer, (iii) enteric coating layer, and (iv) overcoating layer.

### Embodiment 9

The solid oral bead of Embodiment <8>, wherein the active layer includes (i) sugar spheres, (ii) capecitabine, (iii) hypromellose 2910 5 cp, (iv) talc, and (v) corn starch.

### Embodiment 10

The solid oral bead of any one of Embodiments <8> to <9>, wherein the intermediate layer includes (i) sucrose, (ii) talc, and (iii) hypromellose 2910 5 cp.

### Embodiment 11

The solid oral bead of any one of Embodiments <8> to <10>, wherein the enteric coating layer includes (i) hypromellose phthalate, (ii) talc, and (iii) triethyl citrate.

### Embodiment 12

The solid oral bead of any one of Embodiments <8> to <11>, wherein the overcoating layer includes opadry AMB II high performance moisture barrier film coating 88A180040.

### Embodiment 13

The oral liquid solution of any one of embodiments <3>, <5>, and <7>, including (i) active ingredient, (ii) preservative, (iii) buffer, pH control agent, (iv) sweetener-solvent, (v) solvent-vehicle, (vi) flavor, (vii) colorant, and (viii) chelating agent.

### Embodiment 14

The oral liquid solution of embodiment <13>, wherein the active ingredient is raloxifene.

### Embodiment 15

The oral liquid solution of embodiment <13>, wherein the active ingredient is anastrozole.

### Embodiment 16

The oral liquid solution of embodiment <13>, wherein the active ingredient is letrozole.

### Embodiment 17

The oral liquid solution of embodiment <16>, wherein the preservative includes at least one of methylparaben and sodium benzoate.

### Embodiment 18

The oral liquid solution of any one of embodiments <13> to <17>, wherein the buffer, pH control agent includes at least one of citric acid and trisodium citrate dihydrate.

### Embodiment 19

The oral liquid solution of any one of embodiments <13> to <18>, wherein the sweetener-solvent includes at least one of sucralose, sorbitol, and sodium saccharin.

### Embodiment 20

The oral liquid solution of any one of embodiments <13> to <19>, wherein the solvent-vehicle includes at least one of propylene glycol, glycerin, and water.

### Embodiment 21

The oral liquid solution of any one of embodiments <13> to <20>, wherein the chelating agent includes edetate disodium dihydrate.

### Embodiment 22

The oral liquid suspension of any one of embodiments <2>, <4>, and <6>, including (i) active ingredient, (ii) preservative, (iii) buffer-pH control agent, (iv) sweetener-solvent, (v) solvent-vehicle, (vi) flavor, (vii) colorant, (viii) anticaking/suspending agent, (ix) viscosifying agent, and (x) taste masking agent.

### Embodiment 23

The oral liquid suspension of embodiment <22>, wherein the active ingredient is raloxifene.

### Embodiment 24

The oral liquid suspension of embodiment <22>, wherein the active ingredient is anastrozole.

### Embodiment 25

The oral liquid suspension of embodiment <22>, wherein the active ingredient is letrozole.

### Embodiment 26

The oral liquid suspension of embodiment <22>, wherein the preservative includes at least one of methylparaben and sodium benzoate.

### Embodiment 27

The oral liquid suspension of any one of embodiments <23> to <26>, wherein the buffer-pH control agent includes at least one of sodium phosphate dibasic and sodium phosphate monobasic.

### Embodiment 28

The oral liquid suspension of any one of embodiments <23> to <27>, wherein the sweetener-solvent includes at least one of saccharin sodium, dihydrate and sorbitol.

### Embodiment 29

The oral liquid suspension of any one of embodiments <23> to <28>, wherein the solvent-vehicle includes at least one of propylene glycol, glycerin, and water.

### Embodiment 30

The oral liquid suspension of any one of embodiments <23> to <29>, wherein the flavor includes natural cherry flavor.

### Embodiment 31

The oral liquid suspension of any one of embodiments <23> to <30>, wherein the colorant includes at least one of FD&C Red No. 40, allura red AC, and FD&C Yellow No. 6.

### Embodiment 32

The oral liquid suspension of any one of embodiments <23> to <31>, wherein the anticaking/suspending agent includes Vivapur MCG 591P.

### Embodiment 33

The oral liquid suspension of any one of embodiments <23> to <32>, wherein the viscosifying agent includes at least one of xanthan gum and polyethylene glycol 400.

### Embodiment 34

The oral liquid suspension of any one of embodiments <23> to <33>, wherein the taste masking agent includes sucralose.

### EXAMPLES

### Example 1: Anastrozole Oral Solution. 0.1 mg/mL

### Components and Composition

| | | | | | Maximum | | | | |
|---|---|---|---|---|---|---|---|---|---|
| | | | | Amount/unit | Daily Dose | | | | |
| Active Ingredient | Grade | UNII | Function | (mg/mL) | (MDD) | | | | |
| Anastrozole | USP | 2Z07MYW1AZ | Active | 0.1 | 1.00 | | | | |
| | | | | | | Maximum | | | |
| | | | | | Total Daily | Daily | | | |
| | | | | Amount/unit | Intake (TDI) | Exposure | | IID | IID |
| Inactive Ingredient | | UNII | Function | (mg) | based on MDD | (MDE) | | Route | Dosage form |
| Citric Acid | USP | XF417D3PSL | Buffer | 2.40 | 24.00 | 531 | mg | Oral | Solution |
| Anhydrous | | | | | | | | | |
| Sorbitol Solution, | USP | 8KW3E207O2 | Sweetener | 250.00 | 2500.00 | 25830 | mg | Oral | Solution |
| 70 Percent | | | | | | | | | |
| Sodium Saccharin | USP | SB8ZUX40TY | Sweetener | 1.50 | 15.00 | 2250 | | Oral | Powder; for |
| | | | | | | | | | suspension |
| Sodium Benzoate, | USP | OJ245FE5EU | Preservative | 1.00 | 10.00 | 120 | mg | Oral | Suspension |
| Powder | | | | | | | | | |
| Methylparaben, | NF | A2I8C7HI9T | Preservative | 1.00 | 10.00 | 346 | mg | Oral | Solution |
| Powder | | | | | | | | | |
| Edetate Disodium | USP | 7FLD91C86K | Chelating | 3.00 | 30.00 | 360 | mg | Oral | Solution |
| Dihydrate | | | Agent | | | | | | |
| FD and C Red | NA | WZB9127XOA | Colorant | 0.01 | 0.10 | 28 | mg | Oral | Solution |
| No. 40 | | | | | | | | | |
| Cherry Flavor, | NA | NA | Flavor | 2.00 | 20.00 | 290 | MG | Oral | Suspension |
| Nat & Art | | | | | | | | | |

| | | | | | | | | |
|---|---|---|---|---|---|---|---|---|
| Purified Water | USP | NA | Solvent | 739.09 | 7390.90 | NA | NA | NA |

### Example 2: Capecitabine Granules for Suspension 25 mg/mL

### Components and Composition

| | | | | |
|---|---|---|---|---|
| Item | | | | |
| No | Ingredient | Function | mg/dose | % w/w |

Dry Blending

| | | | | |
|---|---|---|---|---|
| 1 | Capecitabine | Active | 25.00 | 12.50 |
| 2 | Carboxymethylcellulose | Viscosity | 3.30 | 1.65 |
| | Sodium | Enhancer | | |
| 3 | Xanthan Gum | Suspending Agent | 0.77 | 0.38 |
| 4 | Sucrose | Granulation Aid | 159.36 | 79.68 |
| 5 | Sodium Citrate | Buffering Agent | 10.60 | 5.30 |
| 6 | Citric Acid Monohydrate | Buffering Agent | 0.02 | 0.01 |
| 7 | FD&C Red No 40 | Colorant | 0.04 | 0.02 |

Granulation Solution

| | | | | |
|---|---|---|---|---|
| 8 | Carboxymethylcellulose | Viscosity | 0.30 | 0.15 |
| | Sodium | Enhancer | | |
| 9 | Xanthan Gum | Suspending Agent | 0.21 | 0.11 |
| 10 | Water | Granulation Aid | 13.00 | q.s. |

Final Blending

| | | | | |
|---|---|---|---|---|
| 11 | Cherry Flavor | Flavor | 0.40 | 0.20 |

| | | |
|---|---|---|
| Total | 200.00 | 100.00 |

### Manufacturing Process

The following was the manufacturing process,

1. In a glass beaker, weigh Water (ingredient #10).

2. To this, add ingredients in following order. Mix for at least 30 minutes.
Xanthan Gum (Ingredient #9)
Carboxymethylcellulose Sodium (Ingredient #8)

3. Divide Sucrose in 2 equal parts.

4. Add ingredients in high shear granulator in following order.
Sucrose Part A (Ingredient #4A)
Capecitabine (Ingredient #1)
Carboxymethylcellulose (Ingredient #2)
Xanthan Gum (Ingredient #3)
Sodium Citrate (Ingredient #5)
Citric Acid Monohydrate (Ingredient #6).FD&C Red No 40 (Ingredient #7)
Sucrose Part B (Ingredient #4B)

5. Blend all the materials for 10 mins at 100 rpm impeller speed.

6. Add solution from step 2 into the blend from step 5. Spray rate-20-25 mins and Impeller speed-400 rpm.

7. At the end of granulation, open the bowl and scrap the walls.

8. Knead the granules for 1 min at 500 rpm impeller speed.

9. Pass the granules through 0.062 inch screen using comil. Measure LOD

10. Dry the granules at 50-60° C. using oven until LOD is NMT 2.5%

11. Pass the granules through 0.045 inch screen using comil.

12. Add the granules and Cherry Flavor in V-Blender and mix for 10 mins at 24 rpm.

13. Provide samples to lab for testing

### Example 3: Letrozole Oral Suspension. 0.25 mg/mL

### Components and Composition

| | | | | | Maximum | | | | |
|---|---|---|---|---|---|---|---|---|---|
| | | | | Amount/unit | Daily Dose | | | | |
| Active Ingredient | Grade | UNII | Function | (mg) | (MDD) | | | | |
| Letrozole | USP | 7LKK855W8I | Active | 0.25 | 2.5 | | | | |
| | | | | | | Maximum | | | |
| | | | | | Total Daily | Daily | | | |
| | | | | Amount/unit | Intake (TDI) | Exposure | | IID | IID |
| Inactive Ingredient | | UNII | Function | (mg) | based on MDD | (MDE) | | Route | Dosage form |
| Methylparaben, NF | NF | A2I8C7HI9T | Preservative | 1.00 | 10 | 40 | mg | Oral | Suspension, |
| | | | | | | | | | Extended |
| | | | | | | | | | Release |
| Sodium Benzoate, | NF | OJ245FE5EU | Preservative | 0.30 | 3 | 120 | mg | Oral | Suspension |
| Powder, NF | | | | | | | | | |
| Saccharin Sodium, | USP | SB8ZUX40TY | Sweetener | 0.75 | 7.5 | 2250 | mg | Oral | Powder; For |
| Dihydrate, Powder, USP | | | | | | | | | Suspension |
| Sodium Phosphate | USP | 22ADO53M6F | Buffer | 1.60 | 16 | 210 | mg | Oral | Tablet; |
| Dibasic | | | | | | | | | Extended |
| | | | | | | | | | Release |
| Sodium Phosphate | USP | KH7I04HPUU | Buffer | 0.27 | 2.7 | 65 | mg | Oral | Suspension |
| Monobasic | | | | | | | | | |
| Sorbitol Solution, | USP | 8KW3E207O2 | Sweetener | 30.00 | 300 | 25830 | mg | Oral | Solution |
| 70 Percent, USP | | | | | | | | | |
| Propylene Glycol, USP | USP | 6DC9Q167V3 | Cosolvent | 22.50 | 225 | 27120 | mg | Oral | Suspension |
| Glycerine 99.7% | USP | PDC6A3C0OX | Solvent | 50.00 | 500 | 26208 | mg | Oral | Suspension |
| PROSOLV ® SMCC | NF | 212693817 | Anticaking | 12.60 | 126 | 3195 | mg | Oral | Capsule |
| 50 (Silicified | | | | | | | | | |
| Microcrystalline | | | | | | | | | |
| Cellulose) NF | | | | | | | | | |
| Carboxymethylcellulose | USP | K679OBS311 | Suspending | 2.00 | 20 | 450 | mg | Oral | Suspension |
| Sodium, Medium Viscosity, | | | Agent | | | | | | |
| Viscosity of 2 Percent | | | | | | | | | |
| Aqueous Solution @ | | | | | | | | | |
| 25 DEG C.: 400-800 | | | | | | | | | |
| cP, USP | | | | | | | | | |
| Xanthan Gum, NF | NF | TTV12P4NEE | Viscosifying | 2.00 | 20 | 600 | mg | Oral | Powder; For |
| | | | Agent | | | | | | suspension |
| Cherry Flavor, Nat | NA | NA | Flavor | 2.00 | 20 | 290 | MG | Oral | Suspension |
| & Art | | | | | | | | | |
| FD and C Red No. 40 | NA | WZB9127XOA | Colorant | 0.02 | 0.2 | 28 | mg | Oral | Solution |
| FD and C Yellow No. 6 | NA | H77VEI93A8 | Colorant | 0.00 | 0 | 28 | mg | Oral | Powder, For |
| | | | | | | | | | Solution |
| Purified Water, USP | USP | NA | Solvent | 823.71 | 8237.1 | NA | | NA | NA |
| Polyethylene Glycol | NF | B697894SG | Viscosifying | 50.00 | 500 | 24000 | mg | Oral | Solution |
| 400, NF | | | Agent | | | | | | |
| Sucralose, NF | NF | 96K6UQ3ZD4 | Taste Masking | 1.00 | 10 | 264 | mg | Oral | Suspension |
| | | | Agent | | | | | | |

### Manufacturing Process Outline:

1. Weighing
The raw materials are weighed.

2. Preparation of Preparation 1
Methylparaben is dissolved in propylene glycol until clear solution.

3. Preparation of Preparation 2
Xanthan gum and Carboxymethylcellulose Sodium are added in water and mixed until completely dissolved. Prosolv SMCC 50 is added and mixed until uniformly dispersed. This is followed by addition of Sodium Benzoate, Sodium Phosphate Dibasic, Sodium Phosphate Monobasic, Saccharin Sodium Dihydrate, Polyethylene glycol 400 and Sorbitol Solution 70% with continuous mixing. Letrozole is added into suspension with continuous mixing.

4. Addition of Preparation 1 and remaining excipients into Preparation 2
Solution from Preparation 1 is added into Preparation 2 with continuous mixing. This is followed by addition of glycerin. Colors, Flavor and Sweetener are added and mixed for 10 minutes.

5. Packaging
At the end of the manufacturing process, Trazodone Oral Suspension is packed into the primary packaging.

### Physical Properties

Appearance—The product is Pink, well-dispersed suspension with cherry odor
pH—between 7.0-7.5
Specific gravity—1.003 g/mL
Viscosity—<400 cP

### Example 4: Raloxifene Hydrochloride Oral Suspension, 5 mg/mL

### Components and Composition

| | | | | | Maximum | | | | |
|---|---|---|---|---|---|---|---|---|---|
| | | | | Amount/unit | Daily Dose | | | | |
| Active Ingredient | Grade | UNII | Function | (mg) | (MDD) | | | | |
| Raloxifene | USP | 4F86W47BR6 | Active | 5.39 | 64.68 | | | | |
| Hydrochloride | | | | | | | | | |
| | | | | | | Maximum | | | |
| | | | | | Total Daily | Daily | | | |
| | | | | Amount/unit | Intake (TDI) | Exposure | | IID | IID |
| Inactive Ingredient | | UNII | Function | (mg) | based on MDD | (MDE) | | Route | Dosage form |
| Methylparaben, NF | NF | A2I8C7HI9T | Preservative | 1.00 | 12.00 | 40 | mg | Oral | Suspension, |
| | | | | | | | | | Extended |
| | | | | | | | | | Release |
| Sodium Benzoate, | NF | OJ245FE5EU | Preservative | 0.30 | 3.60 | 120 | mg | Oral | Suspension |
| Powder, NF | | | | | | | | | |
| Saccharin Sodium, | USP | SB8ZUX40TY | Sweetener | 0.75 | 9.00 | 2250 | mg | Oral | Powder; For |
| Dihydrate, Powder, USP | | | | | | | | | Suspension |
| Sodium Phosphate | USP | 22ADO53M6F | Buffer | 1.60 | 19.20 | 210 | mg | Oral | Tablet; |
| Dibasic | | | | | | | | | Extended |
| | | | | | | | | | Release |
| Sodium Phosphate | USP | KH7I04HPUU | Buffer | 0.27 | 3.24 | 65 | mg | Oral | Suspension |
| Monobasic | | | | | | | | | |
| Sorbitol Solution, | USP | 8KW3E207O2 | Sweetener | 30.00 | 360.00 | 25830 | mg | Oral | Solution |
| 70 Percent, USP | | | | | | | | | |
| Propylene Glycol, USP | USP | 6DC9Q167V3 | Cosolvent | 22.50 | 270.00 | 27120 | mg | Oral | Suspension |
| Glycerin 99.7% | USP | PDC6A3C0OX | Solvent | 50.00 | 600.00 | 26208 | mg | Oral | Suspension |
| PROSOLV ® SMCC | NF | 212693817 | Anticaking | 12.60 | 151.20 | 3195 | mg | Oral | Capsule |
| 50 (Silicified | | | | | | | | | |
| Microcrystalline | | | | | | | | | |
| Cellulose) NF | | | | | | | | | |
| Carboxymethylcellulose | USP | K679OBS311 | Suspending | 2.00 | 24.00 | 450 | mg | Oral | Suspension |
| Sodium, Medium Viscosity, | | | Agent | | | | | | |
| Viscosity of 2 Percent | | | | | | | | | |
| Aqueous Solution @ | | | | | | | | | |
| 25 DEG C.: 400-800 | | | | | | | | | |
| cP, USP | | | | | | | | | |
| Xanthan Gum, NF | NF | TTV12P4NEE | Viscosifying | 2.00 | 24.00 | 600 | mg | Oral | Powder; For |
| | | | Agent | | | | | | suspension |
| Cherry Flavor, | NA | NA | Flavor | 2.00 | 24.00 | 290 | MG | Oral | Suspension |
| Nat & Art | | | | | | | | | |
| FD and C Red No. 40 | NA | WZB9127XOA | Colorant | 0.02 | 0.24 | 28 | mg | Oral | Solution |
| FD and C Yellow No. 6 | NA | H77VEI93A8 | Colorant | 0.00 | 0.02 | 28 | mg | Oral | Powder, For |
| | | | | | | | | | Solution |
| Purified Water, USP | USP | NA | Solvent | 820.00 | 9840.00 | NA | | NA | NA |
| Polyethylene | NF | B697894SGQ | Viscosifying | 50.00 | 600.00 | 24000 | mg | Oral | Solution |
| Glycol 400, NF | | | Agent | | | | | | |
| Sucralose, NF | NF | 96K6UQ3ZD4 | Taste | 1.00 | 12.00 | 264 | mg | Oral | Suspension |
| | | | Masking | | | | | | |
| | | | Agent | | | | | | |

### Manufacturing Process Outline:

1. Weighing
The raw materials are weighed.

2. Preparation of Preparation 1
Methylparaben is dissolved in propylene glycol until clear solution.

3. Preparation of Preparation 2
Xanthan gum and Carboxymethylcellulose Sodium are added in water and mixed until completely dissolved. Prosolv SMCC 50 is added and mixed until uniformly dispersed. This is followed by addition of Sodium Benzoate, Sodium Phosphate Dibasic, Sodium Phosphate Monobasic, Saccharin Sodium Dihydrate, Polyethylene glycol 400 and Sorbitol Solution 70% with continuous mixing. Raloxifene Hydrochloride is added into suspension with continuous mixing.

4. Addition of Preparation 1 and remaining excipients into Preparation 2
Solution from Preparation 1 is added into Preparation 2 with continuous mixing. This is followed by addition of glycerin. Colors, Flavor and Sweetener are added and mixed for 10 minutes.

5. Packaging
At the end of the manufacturing process, Trazodone Oral Suspension is packed into the primary packaging.

### Physical Properties

Appearance—The product is Pink, well-dispersed suspension with cherry odor
pH—between 7.0-7.5
Specific gravity—1.013 g/mL
Viscosity—<400 cP

### Example 5: Raloxifene Hydrochloride Oral Solution, 0.30 mg/mL

### Components and Composition

| | | | | | Maximum | | | | |
|---|---|---|---|---|---|---|---|---|---|
| | | | | Amount/unit | Daily Dose | | | | |
| Active Ingredient | Grade | UNII | Function | (mg/mL) | (MDD) | | | | |
| Raloxifene | USP | 4F86W47BR6 | Active | 0.3 | 60 | | | | |
| Hydrochloride | | | | | | | | | |
| | | | | | | Maximum | | | |
| | | | | | Total Daily | Daily | | | |
| | | | | Amount/unit | Intake (TDI) | Exposure | | IID | IID |
| Inactive Ingredient | | UNII | Function | (mg) | based on MDD | (MDE) | | Route | Dosage form |
| Citric Acid | USP | XF417D3PSL | Buffer | 4.28 | 856 | 720 | mg | Oral | Solution |
| Anhydrous | | | | | | | | | |
| Trisodium Citrate | USP | B22547B95K | Buffer | 1.07 | 214 | 750 | mg | Oral | Solution |
| Dihydrate | | | | | | | | | |
| Sorbitol | USP | 8KW3E207O2 | Sweetener | 107 | 21400 | 26400 | mg | Oral | Solution |
| Solution 70% | | | | | | | | | |
| Glycerin | USP | PDC6A3C0OX | Co-Solvent | 107 | 21400 | 32400 | mg | Oral | Solution |
| Propylene Glycol | USP | 6DC9Q167V3 | Co-Solvent | 21.4 | 4280 | 22602 | mg | Oral | Solution |
| Sodium Benzoate | NF | OJ245FE5EU | Preservative | 1.07 | 214 | 660 | mg | Oral | Solution |
| Sucralose | NF | 96K6UQ3ZD4 | Sweetener | 10.7 | 2140 | 800 | mg | Oral | Solution |
| F&C Red No 40 | NA | WZB9127XOA | Colorant | 0 | 0 | 28 | mg | Oral | Solution |
| Chery Flavor | NA | NA | Flavor | 2.14 | 428 | 356 | mg | Oral | Solution |

| | | | | | | | | |
|---|---|---|---|---|---|---|---|---|
| Purified Water | USP | NA | Solvent | 788.59 | 157718 | NA | Oral | Solution |

### Manufacturing Process Outline

Main tank mixing

1. Addition of purified water and mix at 200-300 rpm.

2. Addition of following ingredients and mixing until dissolved
Citric Acid Anhydrous
Sodium Citrate
Sodium Benzoate
Sorbitol Solution 70%
Propylene Glycol
Glycerin
Sucralose
Cherry Flavor

3. Addition of Raloxifene Hydrochloride with continuous mixing and completely dissolved. Q.S the solution with purified water with continuous mixing for 30 minutes.

4. Filter the solution through 70 micron filter and filled (236.5 mL solution) in 8 oz HDPE bottle with caps and labeled.

### Physical Properties

Appearance—The product is an orange to red solution free from visible particulate matters with cherry odor
pH—between 3.0-3.5
Specific gravity—1.043 g/mL
Viscosity—<10 cP

### Example 6: Anastrozole Oral Solution, 0.1 mg/mL

### Components and Composition

| | | | | | Maximum | | | | |
|---|---|---|---|---|---|---|---|---|---|
| | | | | Amount/unit | Daily Dose | | | | |
| Active Ingredient | Grade | UNII | Function | (mg/mL) | (MDD) | | | | |
| Anastrozole | USP | 2Z07MYW1AZ | Active | 0.2 | 1.00 | | | | |
| | | | | | | Maximum | | | |
| | | | | | Total Daily | Daily | | | |
| | | | | Amount/unit | Intake (TDI) | Exposure | | IID | IID |
| Inactive Ingredient | | UNII | Function | (mg) | based on MDD | (MDE) | | Route | Dosage form |
| Citric Acid | USP | XF417D3PSL | Buffer | 2.40 | 12.00 | 531 | mg | Oral | Solution |
| Anhydrous | | | | | | | | | |
| Sorbitol Solution, | USP | 8KW3E207O2 | Sweetener | 250.00 | 1250.00 | 25830 | mg | Oral | Solution |
| 70 Percent | | | | | | | | | |
| Sodium Saccharin | USP | SB8ZUX40TY | Sweetener | 1.50 | 7.50 | 2250 | | Oral | Powder; for |
| | | | | | | | | | suspension |
| Sodium Benzoate, | USP | OJ245FE5EU | Preservative | 1.00 | 5.00 | 120 | mg | Oral | Suspension |
| Powder | | | | | | | | | |
| Methylparaben, | NF | A2I8C7HI9T | Preservative | 1.00 | 5.00 | 346 | mg | Oral | Solution |
| Powder | | | | | | | | | |
| Edetate Disodium | USP | 7FLD91C86K | Chelating | 3.00 | 15.00 | 360 | mg | Oral | Solution |
| Dihydrate | | | Agent | | | | | | |
| FD and C Red | NA | WZB9127XOA | Colorant | 0.01 | 0.05 | 28 | mg | Oral | Solution |
| No. 40 | | | | | | | | | |
| Cherry Flavor, | NA | NA | Flavor | 2.00 | 10.00 | 290 | MG | Oral | Suspension |
| Nat & Art | | | | | | | | | |

| | | | | | | | | |
|---|---|---|---|---|---|---|---|---|
| Purified Water | USP | NA | Solvent | 739.09 | 3695.45 | NA | NA | NA |

### Manufacturing Process Outline:

1. Main tank mixing
a. Addition of purified water and mix at 200-300 rpm.
b. Addition of edetate disodium dihydrate, sodium saccharin, sorbitol solution with continuous mixing until dissolved.

2. Auxiliary tank mixing
a. Addition of purified water and mix at 500-700 rpm. Heat at 65°−75° C.
b. Addition of methylparaben. Mix until dissolved
c. Addition of sodium benzoate. Mix until dissolved

3. Transfer contents of auxiliary tank to main tank. Rinse auxiliary tank with purified water. Continue Mixing at 200-300 rpm and cool to 38°−42° C.

4. Addition of Anastrozole with continuous mixing and completely dissolved.

5. Addition of citric acid anhydrous, FD&C Red No 40, Nat & Art Cherry Flavor with continuous mixing and completely dissolved.

6. Q.S the solution with purified water with continuous mixing for 30 minutes.

7. Filter the solution through 20 mesh and filled (236.5 mL solution) in 8 oz HDPE bottle with caps and labeled.

### Physical Properties

Appearance—The product is an orange to red solution free from visible particulate matters with cherry odor
pH—between 4.0-4.5
Specific gravity—1.005 g/mL
Viscosity—<10 cP

### Example 7: Letrozole Oral Solution. 0.25 mg/mL

### Components and Composition

| | | | | | Maximum |
|---|---|---|---|---|---|
| | | | | | Daily |
| Active | | | | Amount/unit | Dose |
| Ingredient | Grade | UNII | Function | (mg/mL) | (MDD) |
| Letrozole | USP | 7LKK855W8I | Active | 0.10 | 2.5 |

| | | | | | Total Daily |
|---|---|---|---|---|---|
| | | | | | Intake (TDI) |
| Inactive | | | | Amount/unit | based on |
| Ingredient | | UNII | Function | (mg) | MDD |
| Citric Acid | USP | XF417D3PSL | Buffer | 4.28 | 107 |
| Anhydrous | | | | | |
| Trisodium | USP | B22547B95K | Buffer | 1.07 | 26.75 |
| Citrate | | | | | |
| Dihydrate | | | | | |
| Sorbitol | USP | 8KW3E207O2 | Sweetener | 107.00 | 2675 |
| Solution 70% | | | | | |
| Glycerin | USP | PDC6A3C0OX | Co-Solvent | 107.00 | 2675 |
| Propylene | USP | 6DC9Q167V3 | Co-Solvent | 21.40 | 535 |
| Glycol | | | | | |
| Sodium | NF | OJ245FE5EU | Preservative | 1.07 | 26.75 |
| Benzoate | | | | | |
| Sucralose | NF | 96K6UQ3ZD4 | Sweetener | 10.70 | 267.5 |
| F&C Red | NA | WZB9127XOA | Colorant | 0.00 | 0.0535 |
| No 40 | | | | | |
| Chery Flavor | NA | NA | Flavor | 2.14 | 53.5 |
| Purified Water | USP | NA | Solvent | 788.59 | 19714.75 |

### Manufacturing Process Outline

Main tank mixing

1. Addition of purified water and mix at 200-300 rpm.

2. Addition of following ingredients and mixing until dissolved.
Citric Acid Anhydrous
Sodium Citrate.
Sodium Benzoate.
Sorbitol Solution 70%.
Propylene Glycol
Glycerin.
Sucralose.
Cherry Flavor

3. Addition of Letrozole with continuous mixing and completely dissolved. Q.S the solution with purified water with continuous mixing for 30 minutes.

4. Filter the solution through 70 micron filter and filled (236.5 mL solution) in 8 oz HDPE bottle with caps and labeled.

### Physical Properties

Appearance—The product is an orange to red solution free from visible particulate matters with cherry odor
pH—between 3.0-3.5
Specific gravity—1.043 g/mL
Viscosity—<10 cP

### Example 8: Production Example

In following with the disclosure herein, the oncological active ingredients in Table 1 can be formulated as granules, oral liquids, and/or oral suspensions. Formulation as a granule is often desirable when the oncological active ingredient is relatively unstable in aqueous solution.1 In 1 Information regarding the stability in solution of the active ingredient can be obtained from, e.g., the European Medicines Agency Assessment Reports. doing so, the granule is reconstituted with a liquid beverage (e.g., water) prior to administration. Formulation as an oral suspension is often desirable when the active ingredient is relatively insoluble in aqueous solution. Likewise, formulation as an oral liquid is often desirable when the oncological active ingredient is relatively soluble in aqueous solution. Based in part upon the solubility and stability in aqueous solution of the active ingredient, the formulator can select the appropriate dosage form further taking into account, e.g., preferences of the patients. Such preferences can include the desire to use a ready-to-use (RTU) product, to avoid reconstitution. Alternatively, such preferences can include the desire to use granules, which allow the patient to select liquid beverages, other than water, for reconstitution. The formulator can also use the oncological active ingredient in any one or more of the amounts (i.e., strengths) shown in Table 1, and can market the product for any one or more of the indications shown therein.

TABLE 1

| Active | | | | |
|---|---|---|---|---|
| Ingredient² | Strength(s) | | Indication(s) | Solubility |
| tretinoin | 10 | mg | acute promyelocytic leukemia | insoluble in water |
| abemaciclib | 50 mg; 100 mg; | | early breast cancer; advanced | insoluble in water |
| | 150 mg; 200 mg | | or metastatic breast cancer | |
| abiraterone acetate | 250 | mg | metastatic castration-resistant | practically |
| | | | prostate cancer | insoluble in water |
| acalabrutinib | 100 | mg | mantle cell lymphoma; chronic | freely soluble in water at |
| | | | lymphocytic leukemia or small | pH below 3; practically |
| | | | lymphocytic lymphoma | insoluble at pH above 6 |
| afatinib | 40 mg; | | metastatic non-small cell lung | water soluble |
| | 30 mg; 20 mg | | cancer (NSCLC); metastatic, | |
| | | | squamous NSCLC | |
| avapritinib | 100 mg; | | gastrointestinal stromal | insoluble in water |
| | 200 mg; 300 mg | | tumor | |
| everolimus | 2.5 mg; 5 mg; | | breast cancer; neuroendocrine | insoluble in water |
| | 7.5 mg; 10 mg | | tumors; renal cell carcinoma; | |
| | | | renal angiomyolipoma and | |
| | | | tuberous sclerosis complex | |
| alectinib | 150 | mg | lung cancer | insoluble in water |
| melphalan | 2 | mg | multiple myeloma; epithelial | practically |
| | | | carcinoma of the ovary | insoluble in water |
| anastrozole | 1 | mg | breast cancer | moderate aqueous |
| | | | | solubility (0.5 mg/mL |
| | | | | at 25° C.) |
| apalutamide | 60 | mg | prostate cancer | practically |
| | | | | insoluble in water |
| exemestane | 25 | mg | breast cancer | practically |
| | | | | insoluble in water |
| axitinib | 1 mg; 5 mg | | renal cell carcinoma | The solubility of |
| | | | | axitinib in aqueous |
| | | | | media over the range |
| | | | | pH 1.1 to pH 7.8 is in |
| | | | | excess of 0.2 μg/mL |
| azacitidine | 200 mg; 300 mg | | myeloid leukemia | water soluble |
| bexarotene | 75 | mg | cutaneous T-cell lymphoma | insoluble in water |
| bicalutamide | 50 | mg | metastatic carcinoma of the | practically |
| | | | prostate | insoluble in water |
| bosutinib | 100 mg; 500 mg | | chronic myelogenous | highly soluble at or |
| | | | leukemia | below pH 5; reduces |
| | | | | rapidly above pH 5 |
| brigatinib | 180 mg; | | lung cancer | insoluble in water |
| | 90 mg; 30 mg | | | |
| busulfan | 2 | mg | chronic myelogenous leukemia | insoluble in water |
| capecitabine | 150 mg; 500 mg | | colon cancer; colorectal | 6 mg/mL at 25° C. |
| | | | cancer; breast cancer | |
| lomustine | 5 mg; 10 mg; | | brain tumors; Hodgkin's | insoluble in water |
| | 40 mg; 100 mg | | lymphoma | |
| cabozantinib | 20 mg; | | renal cell carcinoma; | practically |
| | 40 mg; 60 mg | | hepatocellular carcinoma | insoluble in water |
| cabozantinib | 20 mg; 80 mg | | medullary thyroid cancer | practically |
| (S)-malate | | | | insoluble in water |
| vandetanib | 100 mg; 300 mg | | medullary thyroid cancer | practically |
| | | | | insoluble in water |
| etoposide | 50 | mg | refractory testicular tumors; | sparingly soluble |
| | | | small cell lung cancer | in water |
| ceritinib | 150 | mg | lung cancer | insoluble in water |
| chlorambucil | 2 | mg | lymphocytic leukemia; | insoluble in water |
| | | | malignant lymphomas | |
| | | | including lymphosarcoma; | |
| | | | giant follicular lymphoma; | |
| | | | Hodgkin's disease | |
| lorlatinib | 25 mg; 100 mg | | lung cancer | solubility decreases over |
| | | | | pH 2.55 to pH 8.02 |
| | | | | from 32.38 mg/mL |
| | | | | to 0.17 mg/mL |
| crizotinib | 200 mg; 250 mg | | lung cancer; anaplastic large | Solubility decreases |
| | | | cell lymphoma; inflammatory | over the range pH 1.6 |
| | | | | to pH 8.2 from |
| | | | myofibroblastic tumor | greater than 10 mg/mL |
| | | | | to less than 0.1 mg/mL |
| dabrafenib | 50 mg; 75 mg | | melanoma | Very slightly soluble |
| | | | | at pH 1; practically |
| | | | | insoluble above pH 4 |
| trametinib | 0.5 mg; 2 mg | | melanoma | practically |
| | | | | insoluble in water |
| dasatinib | 20 mg; 50 mg; | | chronic myeloid leukemia; | insoluble in water |
| | 70 mg; 80 mg; | | lymphoblastic leukemia | |
| | 100 mg; 140 mg | | | |
| midostaurin | 25 | mg | acute myeloid leukemia; | insoluble in water |
| | | | aggressive systemic | |
| | | | mastocytosis, systemic | |
| | | | mastocytosis with associated | |
| | | | hematological neoplasm, or | |
| | | | mast cell leukemia | |
| encorafenib | 75 | mg | melanoma; metastatic | slightly soluble at pH 1, |
| | | | colorectal cancer | very slightly soluble at |
| | | | | pH 2, and insoluble at |
| | | | | pH 3 and higher |
| binimetinib | 15 | mg | melanoma | Slightly soluble at pH 1, |
| | | | | very slightly soluble at |
| | | | | pH 2, and practically |
| | | | | insoluble at pH 4.5 |
| | | | | and higher |
| entrectinib | 100 mg; 200 mg | | non-small cell lung cancer; | insoluble in water |
| | | | solid tumors | |
| enzalutamide | 40 | mg | castration-resistant prostate | practically |
| | | | cancer | insoluble in water |
| erlotinib | 25 mg; | | non-small cell lung cancer; | very slightly |
| | 100 mg; 150 mg | | pancreatic cancer | soluble in water |
| letrozole | 2.5 | mg | breast cancer | practically |
| | | | | insoluble in water |
| fludarabine phosphate | 10 | mg | chronic lymphocytic leukemia | 3 mg/mL |
| flutamide | 125 | mg | metastatic carcinoma of the | insoluble in water |
| | | | prostate; metastatic carcinoma; | |
| | | | prostatic carcinoma | |
| tivozanib | 1.34 | mg | renal cell carcinoma | insoluble in water |
| gefitinib | 250 | mg | non-small cell lung cancer | sparingly soluble at |
| | | | | pH 1, but is practically |
| | | | | insoluble above pH 7, |
| | | | | with the solubility |
| | | | | decreasing sharply |
| | | | | between pH 4 and pH 6 |
| imatinib mesylate | 100 mg; 400 mg | | chronic myeloid leukemia; | soluble in aqueous |
| | | | lymphoblastic leukemia; | buffers less than or |
| | | | myelodysplastic/ | equal to pH 5.5 but is |
| | | | myeloproliferative | very slightly soluble |
| | | | diseases; aggressive | to insoluble in |
| | | | systemic mastocytosis; | neutral/alkaline |
| | | | hypereosinophilic syndrome | aqueous buffers |
| | | | and/or chronic eosinophilic | |
| | | | leukemia; dermatofibrosarcoma | |
| | | | protuberans; gastrointestinal | |
| | | | stromal tumors | |
| topotecan | 0.25 mg; 1 mg | | relapsed small cell lung cancer | water soluble |
| hydroxyurea | 500 | mg | chronic myeloid leukemia; | 15 mg/mL |
| | | | squamous cell carcinomas of | |
| | | | the head and neck | |
| ibandronate sodium | 150 | mg | postmenopausal osteoporosis | water soluble |
| palbociclib | 75 mg; | | breast cancer | At or below pH 4, |
| | 100 mg; 125 mg | | | palbociclib behaves |
| | | | | as a high-solubility |
| | | | | compound. Above pH 4, |
| | | | | the solubility of the |
| | | | | drug substance reduces |
| | | | | significantly |
| ibrutinib | 70 mg; | | mantle cell lymphoma; chronic | practically |
| | 140 mg \| 140 mg; | | lymphocytic leukemia or small | insoluble in water |
| | 280 mg; 420 mg; | | lymphocytic lymphoma; | |
| | 560 mg | | Waldenstrom's | |
| | | | macroglobulinemia; marginal | |
| | | | zone lymphoma; chronic graft | |
| | | | versus host disease | |
| ponatinib | 15 mg; 45 mg | | chronic myeloid leukemia; | The solubility of |
| | | | acute lymphoblastic | ponatinib in pH 1.7, 2.7, |
| | | | leukemia | and 7.5 buffers is 7790 |
| | | | | mcg/ml, 3.44 mcg/ml, |
| | | | | and 0.16 mcg/ml, |
| | | | | respectively, indicating |
| | | | | a decrease in solubility |
| | | | | with increasing pH |
| ixazomib | 2.3 mg; 3 mg; 4 mg | | multiple myeloma | The solubility of |
| | | | | ixazomib citrate in 0.1N |
| | | | | HCl (pH 1.2) at 37° C. |
| | | | | is 0.61 mg/mL (reported |
| | | | | as ixazomib). The |
| | | | | solubility increases |
| | | | | as the pH increases |
| lenalidomide | 2.5 mg; 5 mg; | | multiple myeloma; | insoluble in water |
| | 10 mg; 15 mg; | | transfusion-dependent anemia; | |
| | 20 mg; 25 mg | | mantle cell lymphoma; | |
| | | | follicular lymphoma; marginal | |
| | | | zone lymphoma | |
| ribociclib | 200 | mg | breast cancer | insoluble in water |
| larotrectinib | 25 mg; 100 mg | | solid tumors | The aqueous solubility |
| | | | | of larotrectinib at 37° C. |
| | | | | is pH dependent (very |
| | | | | soluble at pH 1.0 and |
| | | | | freely soluble at pH 6.8) |
| lenvatinib | 4 mg; 10 mg | | differentiated thyroid cancer; | slightly soluble in water |
| | | | renal cell carcinoma; | |
| | | | hepatocellular carcinoma; | |
| | | | endometrial carcinoma | |
| trifluridine; tipiracil | 15 mg | | metastatic colorectal cancer | water soluble; water |
| hydrochloride | trifluridine/6.14 mg | | | soluble |
| | tipiracil; 20 mg | | | |
| | trifluridine/8.19 mg | | | |
| | tipiracil | | | |
| olaparib | 100 mg; 150 mg | | ovarian cancer; breast | low solubility |
| | | | cancer; pancreatic cancer; | |
| | | | prostate cancer | |
| mitotane | 500 | mg | adrenal cortical carcinoma | insoluble in water |
| thalidomide | 50 mg; 100 mg; | | multiple myeloma; erythema | sparingly soluble in |
| | 150 mg; 200 mg | | nodosum leprosum | water |
| medroxyprogesterone | 2.5 mg; | | amenorrhea and abnormal | insoluble in water |
| acetate | 5 mg; 10 mg | | uterine bleeding (uterine | |
| | | | cancer); endometrial hyperplasia | |
| methotrexate | 2.5 | mg | acute lymphoblastic leukemia; | insoluble in water |
| | | | mycosis fungoides; | |
| | | | non-Hodgkin lymphoma | |
| neratinib | 40 | mg | breast cancer | Neratinib maleate is |
| | | | | sparingly soluble at pH |
| | | | | 1.2 (32.90 mg/mL) and |
| | | | | insoluble at approximate |
| | | | | pH 5.0 and above |
| | | | | (0.08 mg/mL or less) |
| sorafenib tosylate | 200 | mg | unresectable hepatocellular | practically |
| | | | carcinoma; renal cell | insoluble in water |
| | | | carcinoma; thyroid carcinoma | |
| nilotinib | 150 mg; 200 mg | | chronic myeloid leukemia | The solubility of |
| | | | | nilotinib in aqueous |
| | | | | solutions decreases |
| | | | | with increasing pH |
| nintedanib | 100 mg; 150 mg | | idiopathic pulmonary fibrosis | Nintedanib displays a |
| | | | | pH-dependent |
| | | | | solubility profile with |
| | | | | increased solubility at |
| | | | | acidic pH less than 3 |
| niraparib | 100 | mg | epithelial ovarian, fallopian | Niraparib solubility is |
| | | | tube, or primary peritoneal | pH independent below |
| | | | cancer | the pKa of 9.95, with |
| | | | | an aqueous free base |
| | | | | solubility of 0.7 mg/mL |
| | | | | to 1.1 mg/mL across the |
| | | | | physiological pH range |
| osimertinib | 40 mg; 80 mg | | non-small cell lung cancer | insoluble in water |
| panobinostat | 10 mg; | | multiple myeloma | The aqueous solubility |
| | 15 mg; 20 mg | | | of panobinostat is pH |
| | | | | dependent, with higher |
| | | | | pH resulting in lower |
| | | | | solubility |
| pazopanib | 200 | mg | renal cell carcinoma; soft | insoluble in water |
| | | | tissue carcoma | |
| pemigatinib | 4.5 mg; | | cholangiocarcinoma; | The solubility of |
| | 9 mg; 13.5 mg | | myeloid/lymphoid neoplasms | pemigatinib is pH |
| | | | | dependent with |
| | | | | decreasing solubility |
| | | | | observed with |
| | | | | increasing pH |
| pomalidomide | 1 mg; 2 mg; | | multiple myeloma | low solubility in all |
| | 3 mg; 4 mg | | | pH solutions (about |
| | | | | 0.01 mg/mL) |
| temozolomide | 5 mg; 20 mg; | | glioblastoma multiforme; | 7 mg/mL |
| | 100 mg; 140 mg; | | refractory anaplastic | |
| | 180 mg; 250 mg | | astrocytoma | |
| idelalisib | 100 mg; 150 mg | | chronic lymphocytic leukemia; | pH-dependent aqueous |
| | | | non-Hodgkin lymphoma; small | solubility ranging from |
| | | | lymphocytic lymphoma | |
| raloxifene | 60 | mg | osteoporosis | very slightly |
| hydrochloride | | | | soluble in water |
| regorafenib | 40 | mg | colorectal cancer | practically |
| | | | | insoluble in water |
| recaparib | 200 mg; 250 mg; | | epithelial ovarian, fallopian | pH-independent low |
| | 300 mg | | tube, or primary peritoneal | solubility of |
| | | | cancer | approximately 1 mg/mL |
| | | | | across the physiological |
| | | | | pH range |
| ruxolitinib phosphate | 5 mg; 10 mg; 15 mg; | | myelofibrosis; polycythemia | soluble in aqueous |
| | 20 mg; 25 mg | | vera; graft-versus-host | buffers across a pH |
| | | | disease; | range of 1 to 8 |
| sunitinib malate | 12.5 mg; | | gastrointestinal stromal | The solubility of |
| | 25 mg; 50 mg | | tumor; renal cell carcinoma; | sunitinib malate in |
| | | | pancreatic neuroendocrine | aqueous media over the |
| | | | tumors | range pH 1.2 to pH 6.8 |
| | | | | is in excess of 25 mg/mL |
| tamoxifen citrate | 10 mg; 20 mg | | breast cancer; ductal | 0.5 mg/mL |
| | | | carcinoma in situ | |
| vemurafenib | 240 | mg | melanoma | practically |
| | | | | insoluble in water |
| venetoclax | 10 mg; | | chronic lymphocytic | very low aqueous |
| | 50 mg; 100 mg | | leukemia | solubility |
| erdafitinib | 3 mg; 4 mg; 5 mg | | urothelial carcinoma | slightly soluble to |
| | | | | practically insoluble, |
| | | | | or insoluble in aqueous |
| | | | | media over a wide |
| | | | | range of pH values |
| zanubrutinib | 80 | mg | mantle cell lymphoma; | The aqueous solubility |
| | | | Waldenstrom's | of zanubrutinib is pH |
| | | | macroglobulinemia; marginal | dependent, from very |
| | | | zone lymphoma; chronic | slightly soluble to |
| | | | lymphocytic leukemia or small | practically insoluble |
| | | | lymphocytic lymphoma | |
| lomustine | 10 mg; | | brain tumors; Hodgkin's | relatively |
| | 40 mg; 100 mg | | lymphoma | insoluble in water |
| duvelisib | 15 mg; 25 mg | | chronic lymphocytic leukemia | practically |
| | | | or small lymphocytic | insoluble in water |
| | | | lymphoma; | |
| | | | follicular lymphoma | |
| cobimetinib fumarate | 20 | mg | melanoma | pH dependent solubility |
| cyclophosphamide | 25 mg; 50 mg | | Hodgkin's disease, | water soluble |
| | | | lymphocytic lymphoma, | |
| | | | mixed-cell type lymphoma, | |
| | | | histiocytic lymphoma, Burkitt's | |
| | | | lymphoma; multiple myeloma, | |
| | | | leukemias, mycosis fungoides, | |
| | | | neuroblastoma, adenocarcinoma | |
| | | | of ovary, retinoblastoma, breast | |
| | | | carcinoma; Nephrotic syndrome | |
| glasdegib | 25 mg; 100 mg | | acute myeloid leukemia | 1.7 mg/mL |
| estramustine | 140 | mg | metastatic and/or progressive | water soluble |
| phosphate | | | carcinoma of the prostate | |
| vismodegib | 150 | mg | basal cell carcinoma | The solubility of |
| | | | | vismodegib is pH |
| | | | | dependent with |
| | | | | 0.1 μg/mL at pH 7 and |
| | | | | 0.99 mg/mL at pH 1 |
| mobocertinib | 40 | mg | non-small cell lung cancer | solubility of 152 mg/mL |
| | | | | in pH 1.0 and >17.6 |
| | | | | mg/mL in pH 6.8 |
| | | | | solutions at 37° C. |
| flutamide | 125 | mg | metastatic carcinoma of the | insoluble in water |
| | | | prostate; prostatic carcinoma; | |
| | | | metastatic carcinoma | |
| toremifene citrate | 60 | mg | breast cancer | Water solubility at |
| | | | | 37° C. is 0.63 mg/mL |
| pralsetinib | 100 | mg | non-small cell lung cancer | The solubility of |
| | | | | pralsetinib in aqueous |
| | | | | media decreases over |
| | | | | the range pH 1.99 to |
| | | | | pH 7.64 from |
| | | | | 0.880 mg/mL to |
| enasidenib | 50 mg; 100 mg | | acute myeloid leukemia | Enasidenib is |
| | | | | practically insoluble |
| | | | | (solubility ≤74 mcg/mL) |
| | | | | in aqueous solutions |
| | | | | across physiological pH |
| | | | | range (pH 1.2 and 7.4) |
| decitabine; | 35 mg decitabine/ | | myelodysplastic syndromes | 22.5 mg/mL; unknown |
| cedazuridine | 100 mg cedazuridine | | | |
| fedratinib | 100 | mg | myelofibrosis | pH-dependent aqueous |
| | | | | solubility; it is freely |
| | | | | soluble in the acidic |
| | | | | condition (>100 mg/mL |
| | | | | at pH 1) and practically |
| | | | | insoluble in the |
| | | | | neutral condition |
| | | | | (4 mcg/mL at pH 7.4) |
| selumetinib | 10 mg; 25 mg | | neurofibromatosis type 1 | Selumetinib sulfate is |
| | | | | freely soluble at |
| | | | | pH <1.5, sparingly |
| | | | | soluble in the pH range |
| | | | | at 1.5 to 3 and slightly |
| | | | | soluble at pH >3 |
| adagrasib | 200 | mg | non-small cell lung cancer | The solubility of |
| | | | | adagrasib in the aqueous |
| | | | | media decreases over |
| | | | | the range pH 1.2 to 7.4 |
| | | | | from >262 mg/mL |
| | | | | to <0.010 mg/mL |
| sotorasib | 120 | mg | non-small cell lung cancer | The solubility of |
| | | | | sotorasib in the aqueous |
| | | | | media decreases over |
| | | | | the range pH 1.2 to 6.8 |
| | | | | from 1.3 mg/mL to |
| | | | | 0.03 mg/mL |
| futibatinib | 4 | mg | intrahepatic | insoluble in water |
| | | | cholangiocarcinoma | |
| mercaptopurine | 50 | mg | acute lymphatic leukemia | insoluble in water |
| nilutamide | 150 | mg | metastatic prostate cancer | slightly |
| | | | | soluble in water |
| darolutamide | 300 | mg | prostate cancer | practically |
| | | | | insoluble in water |
| sonidegib | 200 mg capsules | | basal cell carcinoma | practically |
| | (equivalent to 281 | | | insoluble in water |
| | mg of diphosphate | | | |
| | salt of sonidegib | | | |
| relugolix | 120 | mg | prostate cancer | 0.04 mg per mL in |
| | | | | water at 25° C. |
| alpelisib | 50 mg; | | breast cancer | insoluble in water |
| | 150 mg; 200 mg | | | |
| ripretinib | 50 | mg | gastrointestinal stromal | practically |
| | | | tumor | insoluble in water |
| selpercatinib | 40 mg; 80 mg | | non-small cell lung cancer; | The aqueous solubility |
| | | | medullary thyroid cancer; | of selpercatinib is pH |
| | | | thyroid cancer; solid tumors | dependent, from |
| | | | | sparingly soluble at low |
| | | | | pH to practically |
| | | | | insoluble at neutral pH |
| olutasidenib | 150 | mg | acute myeloid leukemia | practically |
| | | | | insoluble in water |
| asciminib | 20 mg; 40 mg | | myeloid leukemia | insoluble in water |
| thioguanine | 40 | mg | acute nonlymphocytic leukemia | insoluble in water |
| capmatinib | 150 mg; 200 mg | | non-small cell lung cancer | Capmatinib |
| | | | | hydrochloride is slightly |
| | | | | soluble in acidic aqueous |
| | | | | solutions at pH 1 and 2 |
| | | | | and of further decreasing |
| | | | | solubility towards |
| | | | | neutral condition |
| talazoparib tosylate | 0.25 mg; 1 mg | | breast cancer | insoluble in water |
| tazemetostat | 200 | mg | epithelioid sarcoma; | slightly soluble in |
| | | | follicular lymphoma | water |
| tepotinib | 225 | mg | non-small cell lung cancer | insoluble in water |
| ivosidenib | 250 | mg | acute myeloid leukemia; | practically |
| | | | locally advanced or metastatic | insoluble in water |
| | | | cholangiocarcinoma | |
| infigratinib phosphate | 25 mg; 100 mg | | advanced or metastatic | adequate solubility in |
| | | | cholangiocarcinoma | water |
| tucatinib | 50 mg; 150 mg | | breast cancer (including brain | insoluble in water |
| | | | metastases); colorectal cancer | |
| pexidartinib | 125 | mg | tenosynovial giant cell tumor | slightly soluble in |
| | | | | water |
| lapatinib | 250 | mg | breast cancer | 0.007 mg/mL |
| umbralisib tosylate | 200 | mg | marginal zone lymphoma; | practically |
| | | | follicular lymphoma | insoluble in water |
| dacomitinib | 15 mg; | | non-small cell lung cancer | insoluble in water |
| | 30 mg; 45 mg | | | |
| pacritinib | 100 | mg | myelofibrosis | insoluble in water |
| belzutifan | 40 | mg | von Hippel-Lindau disease | insoluble in water |
| | | | (with associated renal cell | |
| | | | carcinoma, central nervous | |
| | | | system hemangioblastomas, | |
| | | | or pancreatic neuroendocrine | |
| | | | tumors | |
| gilteritinib | 40 | mg | acute myeloid leukemia | sparingly |
| | | | | soluble in water |
| selinexor | 20 | mg | relapsed or refractory | insoluble in water |
| | | | multiple myeloma | |
| abiraterone acetate | 125 | mg | prostate cancer | practically |
| | | | | insoluble in water |
| megestrol | 40 | mg | palliative treatment of | 2 mcg/mL at 37° C. |
| acetate | | | advanced carcinoma of the | |
| | | | breast or endometrium | |
| vorinostat | 100 | mg | cutaneous T-cell lymphoma | very slightly soluble |
| | | | | in water |
| everolimus | 0.25 mg; 0.5 mg; | | prophylaxis of organ | insoluble in water |
| | 0.75 mg | | rejection: kidney transplant | |
| | | | and liver transplant | |
| conjugated estrogens | 0.3 mg; 0.45 mg; | | breast cancer (palliation | water soluble |
| (sodium estrone | 0.625 mg; | | only); carcinoma of the | |
| sulfate; sodium | 0.9 mg; 1.25 mg | | prostate (palliation only) | |
| equilin sulfate) | | | | |
| estradiol | 0.5 mg; 1 mg; 2 mg | | breast cancer (palliation | insoluble in water |
| | | | only); carcinoma of the | |
| | | | prostate (palliation only) | |
| levothyroxine sodium | 25, 50, 75, 88, 100, | | management of thyroid | insoluble in water |
| | 112, 125, 137, 150, | | cancer | |
| | 175, 200, 300 mcg | | | |
| levothyroxine sodium | 25, 50, 75, 88, 100, | | management of thyroid | insoluble in water |
| | 112, 125, 137, 150, | | cancer | |
| | 175, 200 mcg | | | |
| levothyroxine sodium | 25, 50, 75, 88, 100, | | management of thyroid | insoluble in water |
| | 112, 125, 137, 150, | | cancer | |
| | 175, 200, 300 mcg | | | |
| levothyroxine sodium | 25, 50, 75, 88, 100, | | management of thyroid | insoluble in water |
| | 112, 125, 137, 150, | | cancer | |
| | 175, 200, 300 mcg | | | |
| ondansetron | 4 mg; 8 mg | | prevention of nausea and | water soluble |
| hydrochloride | | | vomiting associated with | |
| | | | emetogenic cancer | |
| | | | chemotherapy and radiotherapy | |

| | | | |
|---|---|---|---|
| aprepitant | 40 mg; | nausea and vomiting | practically |
| | 80 mg; 125 mg | associated with emetogenic | insoluble in water |

cancer chemotherapy

https://www.ema.europa.eu/en/documents/assessment-report/nubeqa-epar-public-assessment-report_en.pdf (see pg. 16).
²Suitable oncological agents can be obtained from, e.g., Cancer Research UK (https://www.cancerresearchuk.org/about-cancer/treatment/drugs); Florida Blue, Blue Cross Blue Shield (https://www.bcbsfl.com/DocumentLibrary/Providers/Content/RxF_ChemoParity.pdf).

1. An oral solution comprising:
(i) anastrozole;
(ii) citric acid;
(iii) sorbitol;
(iv) sodium saccharin;
(v) sodium benzoate;
(vi) methylparaben;
(vii) edetate disodium (EDTA);
(viii) FD & C Red No. 40;
(ix) cherry flavor; and
(x) water.

2. A solid granule for an oral suspension, comprising:
(i) capecitabine;
(ii) carboxymethylcellulose sodium;
(iii) xanthan gum;
(iv) sucrose;
(v) sodium citrate;
(vi) citric acid monohydrate;
(vii) FD&C Red No 40;
(viii) cherry flavor; and
(ix) xanthan gum.

3. An oral suspension comprising:
(i) letrozole;
(ii) methylparaben;
(iii) sodium benzoate;
(iv) saccharin sodium, dihydrate;
(v) sodium phosphate dibasic;
(vi) sodium phosphate monobasic;
(vii) sorbitol;
(viii) propylene glycol;
(ix) glycerin;
(x) silicified microcrystalline cellulose;
(xi) carboxymethylcellulose sodium;
(xii) xanthan gum;
(xiii) cherry flavor,
(xiv) FD & C Red No. 40;
(xv) FD & C Yellow No. 6;
(xvi) water;
(xvii) polyethylene glycol; and
(xviii) sucralose.

4. An oral suspension comprising:
(i) raloxifene;
(ii) methylparaben;
(iii) sodium benzoate;
(iv) saccharin sodium, dihydrate;
(v) sodium phosphate dibasic;
(vi) sodium phosphate monobasic;
(vii) sorbitol;
(viii) propylene glycol;
(ix) glycerin;
(x) silicified microcrystalline cellulose;
(xi) carboxymethylcellulose sodium;
(xii) xanthan gum;
(xiii) cherry flavor;
(xiv) FD & C Red No. 40;
(xv) FD & C Yellow No. 6; and
(xvi) water;
(xvii) polyethylene glycol; and
(xviii) sucralose.

5. A method of treating breast cancer in a human subject, the method comprising administering to the human subject in need thereof an affective amount of the oral solution of claim 1.

6. A method of treating at least one of breast cancer, gastric cancer, and colorectal cancer in a human subject, the method comprising (i) dissolving or suspending the solid granule of claim 2, in an oral liquid and (ii) administering to the human subject in need thereof an affective amount of the oral liquid.

7. A method of treating breast cancer in a human subject, the method comprising administering to the human subject in need thereof an affective amount of the oral suspension of claim 3.

8. A method of at least one of (i) the treatment and prevention of osteoporosis in postmenopausal women and (ii) reducing the risk of breast cancer in those human subjects at high risk, the method comprising administering to the postmenopausal woman or human subject in need thereof an affective amount of the oral suspension of claim 4.

9. The method of claim 5, wherein the administration comprises multiple doses, over a period of up to 1 year.

10. The method of claim 5, wherein the administration comprises multiple doses, over a period of up to 6 months.

11. The method of claim 5, wherein the administration comprises multiple doses, over a period of up to 3 months.

12. The method of claim 5, wherein the administration comprises multiple doses, over a period of up to 1 month.

13. The method of claim 5, wherein the anastrozole is present in 0.1 mg/mL of the oral solution.

14. The method of claim 6, wherein the capecitabine is present in 25 mg/mL of the oral liquid.

15. The method of claim 7, wherein the letrozole is present in 0.25 mg per ml of the oral suspension.

16. The method of claim 8, wherein the raloxifene is present in 5 mg per ml of the oral suspension.
